(19) United States
(12) Patent Application Publication
(10) Pub. No.: US 2004/0229817 A1
(43) Pub. Date: Nov. 18, 2004

(54) Inhibitors of Hepatitis C virus, compositions and treatments using the same

(75) Inventors: Rohit Duggal, San Diego, CA (US); Koleen Jill Herlihy, San Diego, CA (US); Wei Liu, San Diego, CA (US); Amy Karen Patick, Escondido, CA (US); Eiann Sha, San Diego, CA (US); Weidong Hao, San Diego, CA (US)

Correspondence Address: AGOURON PHARMACEUTICALS, INC., 10350 NORTH TORREY PINES ROAD, LA JOLLA, CA, 92037, US

(73) Assignee: AGOURON PHARMACEUTICALS, INC.

(21) Appl. No.: 10/782,679

(22) Filed: Feb. 18, 2004

Related U.S. Application Data
(60) Provisional application No. 60/448,253, filed on Feb. 18, 2003.

(51) Int. Cl. A61K 38/05; A61K 31/4965

(52) U.S. Cl. 514/18; 514/255.05

(57) ABSTRACT

The invention relates to methods of inhibiting HCV viral replication activity comprising contacting an HCV polymerase with a therapeutically effective amount of a hydroxamate MMP inhibitor, and compositions comprising the same.

### FIELD OF THE INVENTION

The invention relates to methods of inhibiting HCV viral replication activity comprising contacting an HCV polymerase with a therapeutically effective amount of a hydroxamate MMP inhibitor. The invention further relates to pharmaceutical compositions containing the hydroxamate MMP inhibitor in a mammal by administering effective amounts of such hydroxamate MMP inhibitor.

### BACKGROUND OF THE INVENTION

Hepatitis C virus (HCV) is a member of the hepacivirus genus in the family Flaviviridae. It is the major causative agent of non-A, non-B viral hepatitis and is the major cause of transfusion-associated hepatitis and accounts for a significant proportion of hepatitis cases worldwide. Although acute HCV infection is often asymptomatic, nearly 80% of cases resolve to chronic hepatitis. The persistent property of the HCV infection has been explained by its ability to escape from the host immune surveillance through hypermutability of the exposed regions in the envelope protein E2 (Weiner et al., Virology 180:842-848 (1991); Weiner et al. Proc. Natl. Acad. Sci. USA 89:3468-3472 (1992)).

HCV is an enveloped RNA virus containing a single-stranded positive-sense RNA genome approximately 9.5 kb in length (Choo et al., Science 244:359-362 (1989)). The RNA genome contains a 5′-nontranslated region (5′ NTR) of 341 nucleotides (Brown et al., Nucl. Acids Res. 20:5041-5045 (1992); Bukh et al., Proc. Natl. Acad. Sci. USA 89:4942-4946 (1992)), a large open reading frame (ORF) encoding a single polypeptide of 3,010 to 3,040 amino acids (Choo et al. (1989), supra;), and a 3′-nontranslated region (3′-NTR) of variable length of about 230 nucleotides (Kolykhalov et al., J. Virol. 70:3363-3371 (1996); Tanaka et al., J. Virol. 70:3307-3312 (1996)).

The 5′ NTR is one of the most conserved regions of the viral genome and plays a pivotal role in the initiation of translation of the viral polyprotein (Bartenschlager (1997), supra). A single long ORF encodes a polyprotein, which is co- or post-translationally processed into structural (core, E1, and E2) and nonstructural (NS2, NS3, NS4A, NS4B, NS5A, and NS5B) viral proteins by either cellular or viral proteinases (Bartenschlager (1997), supra). The 3′ NTR consists of three distinct regions: a variable region of about 38 nucleotides following the stop codon of the polyprotein, a polyuridine tract of variable length with interspersed substitutions of cytidines, and 98 nucleotides (nt) at the very 3′ end which are highly conserved among various HCV isolates. The order of the genes within the genome is: NH₂-C-E1-E2-p7-NS2-NS3-NS4A-NS4B-NS5A-NS5B-COOH (Grakoui et al., J. Virol. 67:1385-1395 (1993)).

Processing of the structural proteins core (C), envelope protein 1 and (E1, E2), and the p7 region is mediated by host signal peptidases. In contrast, maturation of the nonstructural (NS) region is accomplished by two viral enzymes. The HCV polyprotein is first cleaved by a host signal peptidase generating the structural proteins C/E1, E1/E2, E2/p7, and p7/NS2 (Hijikata et al., Proc. Natl. Acad. Sci. USA 88:5547-5551 (1991); Lin et al., J. Virol. 68:5063-5073 (1994)). The NS2-3 proteinase, which is a metalloprotease, then cleaves at the NS2/NS3 junction. The NS3/4A proteinase complex (NS3 being a serine protease and NS4A acting as a cofactor of the NS3 protease), is then responsible for processing at all the remaining sites (Bartenschlager et al., J. Virol. 67:3835-3844 (1993); Bartenschlager (1997), supra). RNA helicase and NTPase activities have also been identified in the NS3 protein. The N-terminal one-third of the NS3 protein functions as a protease, and the remaining two-thirds of the molecule acts as the helicase/ATPase that is thought to be involved in HCV replication (Bartenschlager (1997), supra). NS4A is a cofactor for the NS3 protease and is followed by NS4B, for which the function is unknown. NS5A is a phosphorylated protein and its function is currently unknown. The fourth viral enzyme, NS5B, is an RNA-dependent RNA polymerase (RdRp) and a key component responsible for replication of the viral RNA genome (Lohmann et al., J. Virol. 71:8416-8428 (1997)).

Since persistent infection of HCV is related to chronic hepatitis and eventually to hepatocarcinogenesis, HCV replication is one of the targets to eradicate HCV reproduction and to prevent hepatocellular carcinoma. New treatment approaches for HCV infection include the development of prophylactic and therapeutic vaccines, the identification of interferons with improved pharmacokinetic characteristics, and the discovery of drugs designed to inhibit HCV replication.

Matrix metalloproteinases (“MMPs”) are a family of enzymes, including, but not limited to, collagenases, gelatinases, matrilysin, and stromelysins, which are involved in the degradation and remodelling of connective tissues. These enzymes are found in a number of cell types that are found in or associated with connective tissue, such as fibroblasts, monocytes, macrophages, endothelial cells and metastatic tumor cells. Matrix metalloproteinases degrade the protein components of the extracellular matrix, i.e. the protein components found in the linings of joints, interstitial connective tissue, basement membranes, cartilage and the like. These proteins include collagen, proteoglycan, fibronectin and lamanin.

Hydroxamate compounds are known as MMP inhibitors (see, e.g., U.S. Pat. Nos. 6,465,508; 6,462,042; 6,429,213; 6,365,587; 6,340,691; 6,268,379; 6,228,869; 6,197,795; 6,162,821; 5,977,408; 5,962,481; 5,929,097; 5,861,436; 5,804,593; 5,700,838; and 5,652,262). Each of these patents is herein incorporated by reference in their entirety.

Nonetheless, none of the hydroxamate MMP inhibitors are known to be HCV inhibitors that have desirable or improved physical and chemical properties appropriate for pharmaceutical applications for treating HCV indications.

### SUMMARY OF THE INVENTION

The present invention provides a novel method of interfering with, decreasing or preventing HCV viral replication activity comprising contacting an HCV polymerase with a therapeutically effective amount of a hydroxamate MMP inhibitor.

In one embodiment of the present invention, the hydroxamate MMP inhibitor is administered orally or intravenously.

The present invention also provides a method of treating a condition that is mediated by HCV polymerase in a patient by administering to said patient a pharmaceutically effective amount of a hydroxamate MMP inhibitor.

The present invention also provides a method of targeting MMP inhibition as a means of treating indications caused by HCV infections.

The present invention also provides a method of targeting viral or cellular targets identified by using MMP inhibitors for treating indications caused by HCV infections.

The present invention also provides a method of identifying cellular or viral pathways interfering with the functioning of the members of HCV replication of which could be used for treating indications caused by HCV infections by administering an MMP inhibitor.

The present invention also provides a method of using MMP inhibitors as tools for understanding mechanism of action of other HCV inhibitors.

The present invention also provides a method of using MMP inhibitors for carrying out gene profiling experiments for monitoring the up or down regulation of genes for the purposed of identifying inhibitors for treating indications caused by HCV infections.

The present invention further provides a pharmaceutical composition for the treatment of Hepatitis C virus (HCV) in a mammal containing an amount of hydroxamate MMP inhibitor that is effective in treating HCV and a pharmaceutically acceptable carrier.

In one embodiment of the present invention, the hydroxamate MMP inhibitors are as disclosed in WO 00/04892, having the formula (I):

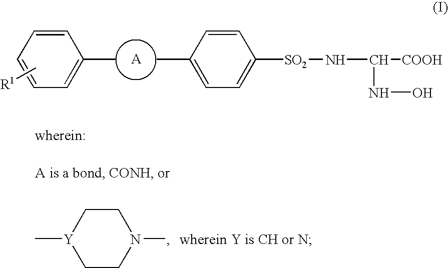

R′ is alkyl, aryl, halo, amino, substituted or distributed amino, or alkoxy; and the pharmaceutically acceptable salts thereof.

In another embodiment of the present invention, the hydroxamate MMP inhibitors are as disclosed in WO 00/04892, having the formula (II):

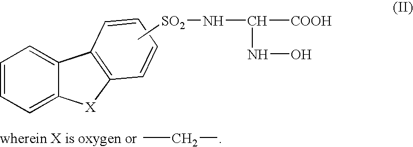

In another embodiment of the present invention, the hydroxamate MMP inhibitors are as disclosed in WO 00/04892, selected from the group consisting of:

2-(2-Phenylethyl)benzoic acid N-hydroxyamide;

2-(Propylthio)-pyridine-3-N-(hydroxy)carboxamide;

[4-(N-Hydroxyamino)-2R-isobutyl-3S-((thien-2-yllthio)methyl)succinyl]-L-phenylalanine-N-methylamide;

N-Hydroxy-5-phenylpentanamide;

2-(Phenyl-2-ethyl)pyridine-3-N-hydroxycarboxamide;

2-(Thiobenzyl)benzoic acid N-hydroxy amide;

6-Biphenyl-4-yl-[2,2-dimethyl-1-(pyridin-4-ylcarbamoyl)-propylcarbamoyl]-hexanoic acid, N-hydroxyamide;

3R(6-(4-Biphenyl)-3-(N-benzylcarbamoyl))-hexanoic acid N-hydroxyamide;

2-Benzylsulfonyl-cyclopent-1-ene-carboxylic acid hydroxamide;

2-Benzylsulfonyl-cyclohex-1-enecarboxylic acid hydroxyamide;

6-Benzylsulfonyl-cyclohex-1-enecarboxylic acid hydroxyamide;

1-(N-Hydroxy)-3-(2-bibenzyl)urea;

3R-(6-(4-Biphenyl)propyl)-N-(3-methylpyridinecarbamoyl)-hexanoic acid N-hydroxy-amide;

4-(2-{[5-Hydroxyamino-3-(3-phenyl-propyl)-3,4-dihydro-2-H-pyrrole-3-carbonyl]-amino}-4-methyl-pentanoylamino)benzoic acid methyl ester;

5-Hydroxyamino-3-(3-phenyl-propyl)-3,4-dihydro-2-H-pyrrole-3-carboxylic acid (2-cyclohexyl-1-methylcarbamoyl-ethyl)amide;

4-(2-{[5-Hydroxyamino-3-(3-pentyl)-3,4-dihydro-2-H-pyrrole-3--carbonyl]amino}-4-methyl-pentanoylamino)benzoic acid methyl ester;

6-Biphenyl-4-yl-3-(R)-(2-hydroxy-1-hydroxymethyl-ethylcarbamoyl)-hexanehydroxamic acid;

6-Biphenyl-4-yl-3(R)-(1(S)-hyroxymethyl-2,2-dimethyl-propylcarbamoyl)-hexanehydroxamicacid;

2-(Biphenyl-4-ylsulfonyl)-cyclohex-1-enecarboxylic acid hydroxyamide;

6-(Biphenyl-4-ylsulfonyl)-cyclohex-1-enecarboxylic acid hydroxyamide;

2-Phenethylsulfanyl-cyclohex-I-enecarboxylic acid hydroxyamide;

2-Benzylsulfanyl-cyclohexancarboxylic acid hydroxamide;

trans-2-Benzylsulfanyl-cyclohexancarboxylic acid hydroxamide;

trans-2-(Biphenyl-4-yl-methylsulfanyl)-cyclohexancarboxylic acid hydroxamide;

6-Biphenyl-4-yl-3-(R)-(1-hydroxymethyl-2-(S)-(1H-imidazol-4-yl)-ethylcarbamoyl)-hexanehydroxamic acid;

N-Hydroxy-2-[2-Oxo-3-(3-phenyl-propyl)-tetrahydro-furan-3-yl]-acetamide;

trans-2-(4-Phenoxy-benzylsulfanyl)-cyclohexancarboxylic acid hydroxamide;

2-(4-Indol-1--yl-benzylsulfanyl)-cyclohexancarboxylic acid hydroxamide;

2-(3-Biphenyl-4-yl-propyl)-N4-hydroxy-N1-(2,4,5-trihydroxy-6-hydroxymethyl-tetrahydro-pyran-3-yl)-succinamide;

2-(2-Biphenyl-4-yl-ethylsulfanyl)-cyclohexane carboxylic acid hydroxyamide;

2-(3-Biphenyl-4-yl-propyl)-N4-hydroxy-N1-(2-hydroxy-cyclohexyl)-succinamide;

6-Biphenyl-4-yl-3-(1-hydroxyimino-ethyl)-hexanoic acid hydroxyamide;

3-(R)-(2-Hydroxy-1-(S)-(1H-imidazol-4-yl)-ethylcarbamoyl)-6-(4-(2-methyl-thiazol-4-yl)-phenyl)-hexanehydroxamic acid;

6-Biphenyl-4-yl-3-(3-hydroxy-piperidine-1-carbonyl)-hexanoic acid-hydroxyamide;

1-(4-Methoxy-benzenesulfonyl)-piperidine-2-carboxylic acid hydroxamide;

1-1-[4-Bromo-phenoxy)-benzenesulfonyl)-piperidine-2-carboxylic acid hydroxyamide;

N-(1-benzyl-2-hydroxy-ethyl)-N4-hydroxy-2-isobutyl-succinamide;

6-Biphenyl-4-yl-3 (R)-2(S)-hydroxy-(1(S)-hydroxymethyl-2,2-dimethyl-propylcarbamoyl)-hexanoic hydroxamic acid;

6-Biphenyl-4-yl-3-(2-hydroxy-1hydroxmethyl-propylcarbamoyl)-hexanoic hydroxamic acid;

trans-2-(3-Biphenyl-4-yl-propyl)-cyclohexane carboxylic acid hydroxyamide;

1-[4-Biphenyl-4-yloxy)-benzenesulfonyl)-piperidine-2-carboxylic acid hydroxamide;

1-(4-Phenoxy-benzenesulfonyl)-piperidine-2-carboxylic acid hydroxamide;

6-Biphenyl-4-yl-3-(R)-(1-(S)-hydroxymethyl-2-(3-pyridyl)-ethylcarbamoyl)-hexanehydroxamic acid;

6-Biphenyl-4-yl-2S-hydroxy-3R-(1S-hydroxymethyl-3-methylsulfanyl-propylcarbamoyl)-hexanoic hydroxamic acid;

1-[-[4-(4-Bromo-phenoxy)-benzenesulfonyl]-4-(tertbutoxycarbonyl)-piperazine-2-carboxylic acid hydroxyamide;

1-[4-(4-Bromo-phenoxy)-benzenesulfonyl]-piperazine-2-carboxylic acid hydroxyamide;

4-Acetyl-1-[4-phenoxy-benzenesulfonyl]-piperazine-2-carboxylic acid, N-hydroxyamide;

1-(Diphenylphosphinic)-piperidine-2-carboxylic acid hydroxamide;

6-Biphenyl-4-yl-3-(R)--(2-oxo-I-tetrahydrofuran-3-(S)-ylcarbamoyl)-hexane hydroxamic acid;

1-[-[4-(4-Bromo-phenoxy)-benzenesulfonyl]-4-methyl-piperazine-2-carboxylic acid N-hydroxyamide;

4-(4-Methoxy-benzenesulfonyl)-thiomorpholine-3-carboxylic acid hydroxyamide;

3-(Diphenylphosphinic)-propanoic acid hydroxyamide;

1-[4-(4-Chlorophenoxy)benzenesulfonyl]-thiomorpholine-3-carbamoyl)piperazine-2-carboxamide;

4[4-Phenoxy-benzenesulfonyl]-piperazine-2-carboxylic acid, N-hydroxyamide;

4[4-Phenoxy-benzenesulfonyl]-thiomorpholine-3-carboxylic acid N-hydroxyamide;

3[2-Biphenyl-4-yl-ethylsulfanyl]-tetrahydro-pyran-4-carboxylic acid N-hydroxyamide;

1-[4-Phenoxy-benzenesulfonyl]-4-methyl-piperazine-2-carboxylic acid N-hydroxyamide;

6-Biphenyl-4-yl-3-(R)-(2-oxo-azepan-3-(S)-ylcarbamoyl)-hexane hydroxamic acid;

4-(1H-Indole-2-sulfonyl)-thiomorpholine-3-carboxylic acid hydroxyamide;

1-(Methyl-phenylphosphinic)-piperidine-2-(R)-carboxylic acid hydroxamide;

1-(1,3-Dihydro-isoindole-2-sulfonyl)-piperidine-2-carboxylic acid hydroxamide;

4-Methyl-1-(4-(4-chlorophenyl)benzenesulfonyl)-N-hydroxy-2R-piperazinecarboxamide hydrochloride;

1-[4-Chlorophenoxybenzenesulfonyl]-N-hydroxy-2R-piperazinecarboxamide;

2-(3-Phenyl-propylsulfonyl)-cyclohexane carboxylic acid hydroxamide;

1-(Pyrolidine-1-sulfonyl)-piperidine-2-carboxylic acid hydroxyamide;

1-(Piperidine-1-sulfonyl)-piperidine-2-carboxylic acid hydroxyamide;

4-[-[4-Bromo-phenoxy-benzenesulfonyl]-oxothiomorpholine-3-carboxylic acid-N-hydroxyamide;

1-[4-(4-Methoxy-phenylsulfanyl)-benzenesulfonyl]-piperdine-2-carboxylic acid hydroxyamide;

1-[4-(4-Cyano-phenoxy)-benzenesulfonyl]-4-(tert-butoxycarbonyl)-piperazine-2-carboxylic acid N-hydroxyamide;

6-Oxo-3-(4-phenoxy-benzenesulfonyl)-hexahydro-pyrimidine-4-carboxylic acid hydroxamate;

4-(t-Butoxycabonyl)-1-(4-(pyridin-2-yl)oxybenzensulfonyl)-N-hydroxy-piperazine-2-carboxamide;

4-[(4-Fluorophenoxy)-benzenesulfonyl]-thiomorpholine-3-carboxylic acid N-hydroxyamide;

4-[4-(Fluoro-phenoxy)-benzenesulfonyl]-oxothiomorpholine-3-carboxylic acid N-hydroxyamide;

4-(4-Butoxy-benzenesulfonyl)-thiomorpholine-3-carboxylic acid hydroxyamide;

4-(4-Butoxy-benzenesulfonyl)-1-oxothiomorpholine-3-carboxylic acid hydroxyamide;

1-[4-(4-Fluorophenyl)benzenesulfonyl]-4-(tert-butoxycarboxyl)2R-piperazine-2-carboxylic acid hydroxyamide;

1-((4-(4-Chlorophenyl)-piperazine)-I-sulfonyl)-piperidine-2carboxylic acid hydroxamide;

cis-2-Phenethylsulfanyl-cyclohexanecarboxylic acid hydroxyamide;

1-[-[4-(4-Fluorophenyl)benzenesulfonyl)-N-hydroxy-2R-piperazinecarboxamide hydrochoride;

1-(Diphenylphosphinic)-pyrolidine-2(R)-carboxylic acid hydroxyamide;

trans-2-Phenethylsulfonyl-cyclohexanecarboxylic acid hydroxyamide;

1-[4-(4-Flourophenyl)-piperazine-1-sulfonyl]-piperidine-2-carboxylic acid hydroxamide;

1-1-[4-(4-Fluorophenylsulfanyl)-benzenesulfonyl]-piperidine-2-carboxylic acid hydroxyamide;

4-1-[4-(Bromo-phenoxy)-benzenesulfonyl]-2,2-dimethyl-1-oxo-thiomorpholine-3-carboxylic acid hydroxyamide;

1-(Pyrrolidine-1-carbonyl)-pyrrolidine-2(R)-carboxylic acid hydroxyamide;

R-4-[4-(Bromophenoxy)-benzenesulfonyl]-2,2-dimethyl-1-oxo-thiomorpholine-3-carboxylic acid hydroxyamide; 4-(Ethoxycarbonyl)methyl-1-(4-(4-chlorophenyl)benzenesulfonyl)-N-hydroxy-2R-piperazinecarboxamide hydrochloride;

1-Phenethylcarbamoyl-pyrrolidine-2-(R)-carboxylic acid hydroxyamide;

1-(4-Benzyl-piperazine-1-sulfonyl)-piperidine-2-carboxylic acid hydroxyamide;

3(S)—N-Hydroxy-4-(4-(pyridin-4-yl)oxybenzenesulfonyl)-2,2-dimethyl-tetrahydro-2H-1,4-thiazine-3-carboxamide;

2(R)4-Methyl-1-(4-(4-fluorophenyl)benzenesulfonyl)-N-hydroxy-piperazine-2-carboxamide;

1-((2-Pyridyl)-4-piperazine-1-sulfonyl)-piperdine-2-carboxylic acid hydroxyamide;

1-1-[4-(Pyridin-4-ylsulfamyl)-benzenesulfonyl]-piperdine-2-carboxylic acid hydroxyamide;

N-(4-Phenoxy-benzenesulfonyl)-D-tert-leucine-N-hydroxyamide;

2,2-Dimethyl-4-[4-(pyridin-2-yloxy)-benzenesulfonyl]-thiomorpholine-3-carboxylic acid hydroxyamide;

N-1-[4-(4-Fluorophenoxyl)benzenesulfonyl)-D-tert-leucine, N-hydroxyamide;

3(R)—N-Hydroxy-4-(4-(pyridin-4-yl)oxybenzenesulfonyl)-2,2-dimethyl-tetrahydro-2H-1,4-thiazine-3-carboxamide hydrochloride;

2-[4-(4-Chloro-phenoxy)-benzenesulfonylamino]-N-hydroxy-3,3-dimethyl-butyramide;

3(R)—N-Hydroxy-4-(4-(fur-3-yl)phenoxybenzenesulfonyl)-2,2-dimethyl-tetrahydro-2H-1,4-thiazine-3-carboxamide;

2-1-[4-(Pyridin-2-yl-oxy)-benzenesulfonylamino]-N-hydroxy-3,3-dimethyl butyramide;

2-(2-Biphenyl-4-yl-ethylsulfonyl)-cyclohex-1-ene-carboxylic acid hydroxyamide;

6-(2-Biphenyl-4-yl-ethyl sulfonyl)-cyclohex-I-ene-carboxylic acid hydroxyamide; N-(4-Phenoxy-benzenesulfonyl)-3,3-dimethyl-S-(methylthio)-D-cysteine, N-hydroxyamide;

1-(4-Phenoxy-piperidine-1-sulfonyl)-piperdine-2-carboxylic acid hydroxyamide;

N-(4-[4-Chlorophenoxy]-benzenesulfonyl)-3,3-dimethyl-S-(methylthio)-D-cysteine, N-hydroxyamide;

N-(4-[4-Chlorophenoxy]-benzenesulfonyl)-3,3-dimethyl-S-(methylsulfoxy)-D-cysteine, N-hydroxyamide;

cis-2-(2-Phenyl-ethanesulfonyl)-cyclohexanecarboxylic acid hydroxyamide;

3(R)—N-Hydroxy-4-(4-(imidazol-1-yl)phenoxybenzenesulfonyl)-2,2-dimethyl-tetrahydro-2H-1,4-thiazine-3-carboxamide;

3(R)—N-Hydroxy-4-(4-(pyridin-4-yl)oxybenzenesulfonyl)-2,2-dimethyl-tetrahydro-2H-1,4-thiazine-3-carboxamide;

4-1-[2-(2-Hydroxycarbamylmethyl-5-phenyl-pentanoylamino)-4-methyl-pentanoyl]-benzoic acid methyl ester;

trans-2-(2-Phenyl-ethanesulfonyl)-cyclohexanecarboxylic acid hydroxyamide;

3,3-Dimethyl-2-(4-phenoxy-phenylsulfanylmethyl)-butyric acid, N-hydroxyamide;

2-(2-Biphenyl-4-yl-ethanesulfonyl)-cyclohexanecarboxylic acid hydroxamate;

2-[-[4-(4-Chlorophenyl)-piperazine-1-sulfonylamino]-3-methyl-3-(pyridin-2ylmethylsulfanyl)-butyric acid N-hydroxyamide;

3,3-Dimethyl-2-(4-phenoxy-phenylsulfonylmethyl)-butyric acid, N-hydroxyamide;

2(R)-[4-(4-Fluoro-phenoxy)benzenesulfonylamino]-3-methyl-3-(pyridin-2-ylsulfanyl)-butyric acid, hydroxyamide;

3(R)—N-Hydroxy-4-(4-(-((pyridin-4-yl)methyl)oxybenzenesulfonyl)--2,2-dimethyl-tetrahydro-2H-1,4-thiazine-3-carboxamide;

1-1-[4-(4-Chloro-phenoxy)-benzenesulfonyl]-4-(I-methyl-1H-imidazole-4-sulfonyl)-piperazine-2-carboxylic acid hydroxamide;

1-[4-(Pyridin-2-ylsulfanyl)-piperidine-I-sulfonyl]-piperidine-2-carboxylic acid hydroxyamide;

2R-[4-(4-Furan-3-yl-phenoxy)-benzenesulfonylamino]-N-hydroxy-3-methyl-3-(pyridin-4-ylsulfanyl)-butyramide;

trans-2-(2-Biphenyl-4-yl-ethylsulfanyl)-cyclohexanecarboxylic acid hydroxyamide;

N4-(2,2-Dimethyl-1S-hydroxymethyl-propyl)-N1-hydroxy-3R[3-(4-pyridin-4-yl-phenyl)-pyrrol-1-yl]-succindiamide;

1-[4-(4-Fluoro-phenoxy)-benzenesulfonyl)]-3,3-dimethyl-5-oxo-piperazine-2-carboxylic acid hydroxyamide;

2(R)-[4-(4-Iodo-phenoxy)benzenesulfonylamino]-3-methyl-(pyridin-3-yl-sulfonyl)butyric acid hydroxyamide;

1-[-[2-(Benzothiazol-2-ylsulfanyl)-piperidine-1-sulfonyl]-piperidine-2-carboxylic acid hydroxyamide;

5-[4-(4-Fluoro-phenoxy)-benzenesulfonyl]-4,5,6,7-tetrahydro-3H-imidazolo[4,5,-c]pyridine-6-carboxylic acid hydroxyamide;

1-[4-(Pyridin-4-ylsulfanyl)-piperidine-1-sulfanyl]-piperidine-2carboxylic acid hydroxyamide;

1-[4-(4-Methoxy-phenylsulfamyl)-piperidine-1-sulfonyl]piperidine-2-carboxylic acid hydroxyamide;

2(R)-[4-(4-Methylphenoxy)benzenesulfonylamino]-3-methyl-3-(pyrdin-3-yl-sulfonyl)butyric acid hydroxyamide;

1-[4-(4-Methyl-phenylsulfamyl)-piperidine-1-sulfonyl]-piperidine-2-carboxylic acid hydroxamide;

4-Methoxy-benzenesulfonyl)-2,2-dimethyl-thiomorpholine-3-carboxylic acid hydroxyamide;

4-1-[4-(4-Chloro-phenoxy)-benzenesulfonyl]-2,2-dimethyl-thiomorpholine-3-carboxylic acid hydroxyamide;

2(R)-[4-(4-bromo-phenoxy)benzenesulfoxylamino]-3-methyl-3-(pyridin-4-yl-sulfoxide)butyric acid hydroxyamide;

4-(4-Methoxy-benzensulfonyl)-2,2-dimethyl-1-oxo-thiomorpholine-3-carboxylic acid hydroxyamide;

4-4-(4-Chloro-phenoxy)-benzenesulfonyl]-2,2-dimethoxy-1-oxo-thiomorpholine-3-carboxylic acid hydroxyamide;

3(S)-2,2-Dimethyl-4-[4-(pyridin-4-ylsulfanyl)-benzenesulfonyl]-thiomorpholine-3-carboxylic acid hydroxyamide;

3,3-Dimethyl-N-hydroxy-2R-[-[4(-(pyridin-4-ylsulfanyl)-piperidine-1-sulfonylamino]-butyramide;

N-Hydroxy-2-[-[(4-methylbenzenesulfonyl)amino]acetamide;

[4(-(4-Imidazol-1-yl-phenoxy)-piperidine-I-sulfonyl]-piperidine-2-carboxylic acid hydroxyamide;

1-[4-(4-Imidazol-1-yl-phenylsulfanyl)-piperidine-1-sulfonyl]-piperidine-2-carboxylic acid hydroxyamide;

2(R)-[4-(4-Chloro-benzoyl)-cyclohexanesulfonyl]-piperidine-1-carboxylic acid hydroxyamide;

1(R)-[4-(4-Chloro-benzoyl)-piperidine-1-sulfonyl]-piperidine-2-carboxylic acid hydroxyamide;

1(R)-(4-Pyridin-2-yl-piperazine-1-sulfonyl)-piperidine-2-carboxylic acid hydroxyamide;

1(R)-[4-(4-Imidazol-1-yl-phenoxy)-piperidine-1-sulfonyl]-piperidine-2-carboxylic acid hydroxyamide;

N-Hydroxy-3,3-dimethyl-2R-[4(-(morpholine-4-carbonyl)-piperidine-1-sulfonylamino]-butyramide;

N-Hydroxy-3-methyl-3-(5-methyl-isoxazol-3-yl-methylsulfanyl)-2R-[4-(pyridin-4-ylsulfanyl)-piperidine-sulfonylamino]-butyramide;

N-Hydroxy-2R-[4-(4-imidazol-1-yl-phenoxy)-piperidine-1-sulfonylamino]-3,3-dimethyl-butyramide;

2R-[4-(4-Chloro-benzoyl)-piperazine-1-sulfonylamino]-N-hydroxy-3-methyl-3-methylsulfanyl-butyramide;

N-Hydroxy-3-methyl-3-methylsulfanyl-2R-[4-(pyridin-4-ylsulfanyl)-piperidine-1-sulfonylamino]-butyramide;

1R,3S,2,2-Dimethyl-1-oxo-4-[-[4(-(pyridin-4-yloxy)-benzenesulfonyl]-thiomorpholine-3-carboxylic acid amide; and the pharmaceutically acceptable salts thereof.

In yet another embodiment of the present invention, the hydroxamate MMP inhibitors are as disclosed in WO 00/04892, selected from the group consisting of:

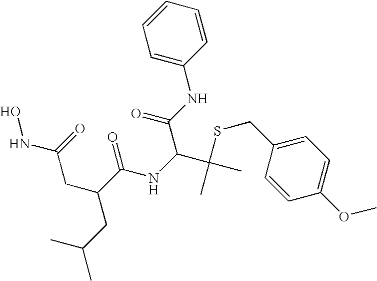

(N¹-{1-(anilinocarbonyl)-2-[(4-methoxybenzyl)thio]-2-methylpropyl}-N⁴-hydroxy-2-isobutylsuccinamide);

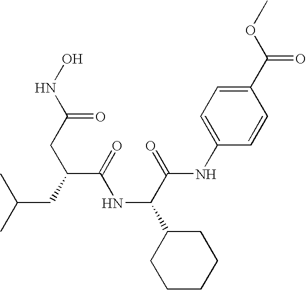

(methyl 4{[(2S)-2-cyclohexyl-2-({(2R)-2-[2-(hydroxyamino)-2-oxoethyl]-4-methylpentanoyl}amino)acetyl]amino}benzoate);

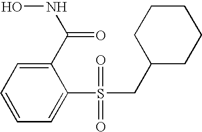

(2-[(cyclohexylmethyl)sulfonyl]-N-hydroxybenzamide);

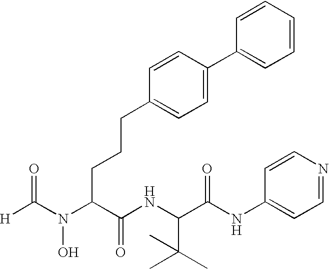

(5-biphenyl-4-yl-N-formyl-N-hydroxynorvalyl-3-methyl-N¹-pyridin-4-ylvalinamide);

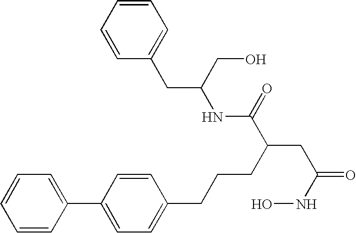

(N¹-(1-benzyl-2-hydroxyethyl)-2-(3-biphenyl-4-ylpropyl)-N⁴-hydroxysuccinamide);

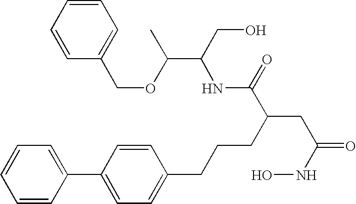

(N¹-[(2-(benzyloxy)-1-(hydroxymethyl)propyl]-2-(3-biphenyl-4-ylpropyl)-N⁴-hydroxysuccinamide);

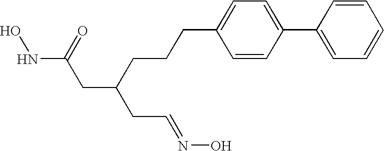

(6-biphenyl-4-yl-N-hydroxy-3-[(2E)-2-(hydroxyimino)ethyl]hexanamide);

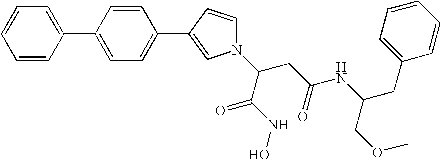

(N⁴-(1-benzyl-2-methoxyethyl)-2-(3-biphenyl-4-yl-1H-pyrrol-1-yl)-N¹-hydroxysuccinamide);

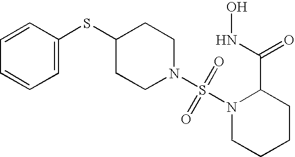

(N-hydroxy-1-{[4-(phenylthio)piperidin-1-yl]sulfonyl}piperidine-2-carboxamide);

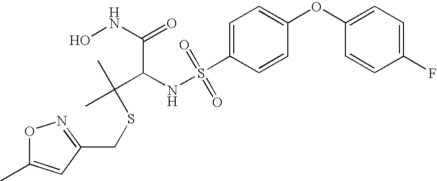

(N²-{[4-(4-fluorophenoxy)phenyl]sulfonyl}-N¹-hydroxy-3{[(5-methylisoxazol-3-yl)methyl]thio}valinamide);

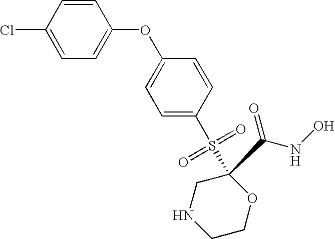

((2R)-2-{[4-(4-chlorophenoxy)phenyl]sulfonyl}-N-hydroxymorpholine-2-carboxamide);

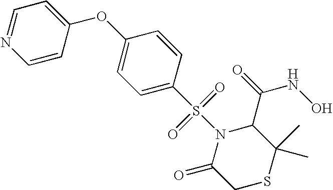

(N-hydroxy-2,2-dimethyl-5-oxo-4-{[4-(pyridin-4-yloxy)phenyl]sulfonyl}thiomorpholine-3-carboxamide); and

the pharmaceutically acceptable salts thereof.

In yet another embodiment of the present invention, the hydroxamate MMP inhibitors as disclosed in U.S. Pat. Nos. 5,753,653 and/or 6,153,757, having the formula (III):

A method according to Claim 1 wherein said hydroxamate MMP inhibitor is of the formula (III):

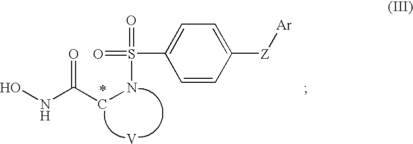

wherein:

Z is O or S; V is a divalent radical which together with C* and N forms a ring having six ring atoms, where each of said ring atoms other than C* and N independently is unsubstituted or substituted by a suitable substituent, and at least one of said other ring atoms is a heteroatom selected from O, N and S, and the remainder are carbon atoms; and Ar is an aryl or heteroaryl group; or a pharmaceutically acceptable prodrug, salt or solvate thereof.

In yet another embodiment of the present invention, the hydroxamate MMP inhibitors are as disclosed in U.S. Pat. No. 5,753,653, selected from the group consisting of:

2(R)—N-hydroxy-1-(4-(4-chlorophenoxy)benzenesulfonyl)-4-(methanesulfonyl)-piperazine-2-carboxamide;

2(R)—N-hydroxy-1-(4-(4-fluorophenoxy)benzenesulfonyl)-4-(methanesulfonyl)-piperazine-2-carboxamide;

3(S)—N-hydroxy-4-(4-((pyrid-4-yl)oxy)benzenesulfonyl)-2,2-dimethyl-tetrahydro-2H-1,4-thiazine-3-carboxamide;

and the pharmaceutically acceptable salts thereof.

In yet another embodiment of the present invention, the hydroxamate MMP inhibitors as disclosed in U.S. Pat. No. 5,985,900, having the formula (IV):

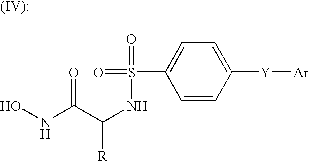

wherein Y is O or S;

Ar is an aryl group or a heteroaryl group;

R is H, an alkyl group, a cycloalkyl group, a heterocycloalkyl group, an aryl group, a heteroaryl group, or —C(O)R¹, wherein R¹ is hydrogen, an alkyl group, a cycloalkyl group, a heterocycloalkyl group, an aryl group, a heteroaryl group, or NR²R³, wherein R² and R³ independently are hydrogen, an alkyl group, a cycloalkyl group, a heterocycloalkyl group, an aryl group, or a heteroaryl group; or the pharmaceutically acceptable salts thereof.

In yet another embodiment of the present invention, the hydroxamate MMP inhibitors are as disclosed in U.S. Pat. No. 5,985,900, selected from the group consisting of:

2(S)—N-hydroxy-3,3-dimethyl-2-[(4-(4-fluorophenoxy)benzenesulfonyl)-amino]butanamide;

2(S)—N-hydroxy-3,3-dimethyl-2-[(4-(4-chlorophenoxy)benzenesulfonyl)-amino]butanamide;

2(S)—N-hydroxy-3-methyl-3-(pyrid-2-yl)methylsulfanyl-2-[(4-(4-fluorophenoxy)benzenesulfonyl)-amino]butanamide;

2(S)—N-hydroxy-3-methyl-3-(pyrid-2-yl)methylsulfanyl-2-[(4-(4-bromophenoxy)-benzenesulfonyl)-amino]butanamide;

2(S)—N-hydroxy-3-methyl-3-(pyrid-2-yl)methylsulfanyl-2-[(4-(4-iodophenoxy)benzenesulfonyl)-amino]butanamide;

2(S)—N-hydroxy-3-methyl-3-(5-methylisoxazol-3-yl)methylsulfanyl-2-[(4-(4-fluorophenoxy)-benzenesulfonyl)amino]butanamide;

2(S)—N-hydroxy-3-methyl-3-(5-methylisoxazol-3-yl)methylsulfanyl-2-[(4-(4-bromophenoxy)-benzenesulfonyl)amino]butanamide;

2(S)—N-hydroxy-3-methyl-3-(pyrid-2-yl)methylsulfanyl-2-[(4-(4-methylphenoxy)-benzenesulfonyl)amino]butanamide;

2(S)—N-hydroxy-3-methyl-3-(5-methylisoxazol-3-yl)methylsulfanyl-2-[(4-(pyrid-4-yloxy)benzenesulfonyl)-amino]butanamide;

2(S)—N-hydroxy-3-methyl-3-(5-methylisoxazol-3-yl)methylsulfanyl-2-[(4-{(pyrid-4-yl)sulfanyl)}-benzenesulfonyl)amino]butanamide;

2(S)—N-hydroxy-3-methyl-3-(1H-imidazol-4-yl)methylsulfanyl-2-[(4-(4-bromophenoxy)benzenesulfonyl)-amino]butanamide;

2(S)—N-hydroxy-3-methyl-3-(1-methyl-1H-imidazol-2-yl)methylsulfanyl-2-[(4-(4-bromophenoxy)-benzenesulfonyl)amino]butanamide;

2(S)—N-hydroxy-3-methyl-3-(1-methyl-1H-imidazol-4-yl)methylsulfanyl-2-[(4-(4-bromophenoxy)-benzenesulfonyl)amino]butanamide;

2(S)—N-hydroxy-3-methyl-3-(4-methyl-4H-[1,2,4]-triazol-3-yl)methylsulfanyl-2-[(4-(4-bromophenoxy)-benzenesulfonyl)amino]butanamide;

2(S)—N-hydroxy-3-methyl-3-(1-methyl-4H-[1,2,4]-triazol-3-yl)methylsulfanyl-2-[(4-(4-bromophenoxy)-benzenesulfonyl)amino]butanamide;

2(S)—N-hydroxy-3-methyl-3-methylsulfanyl-2-[(4-(4-chlorophenoxy)benzenesulfonyl)amino]butananamide; and the pharmaceutically acceptable salts thereof.

In an embodiment of the present invention, the hydroxamate MMP inhibitors is:

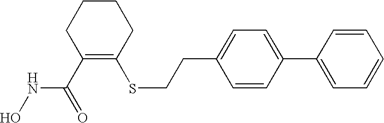

(2-[(2-biphenyl-4-ylethyl)thio]-N-hydroxycyclohex-1-ene-1-carboxamide) or the pharmaceutically acceptable salts thereof.

In another embodiment of the present invention, the hydroxamate MMP inhibitors is:

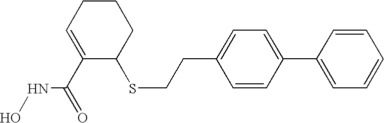

(6-(2-Biphenyl-4-yl-ethylsulfanyl)-cyclohex-1-ene-carboxylic Acid Hydroxyamide) or the pharmaceutically acceptable salts thereof.

In another embodiment of the present invention, the hydroxamate MMP inhibitors is:

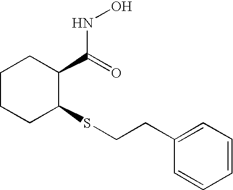

((1R,2R)—N-hydroxy-2-[(2-phenylethyl)thio]cyclohexanecarboxamide), the optically pure compound, enantiomeric mixtures thereof, or the pharmaceutically acceptable salts thereof.

In another embodiment of the present invention, the hydroxamate MMP inhibitors is:

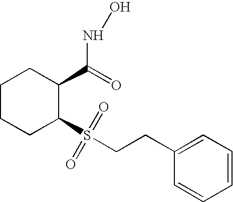

(cis-Phenyl-ethanesulfonyl-cyclohexanecarboxylic Acid Hydroxyamide), the optically pure compound, enantiomeric mixtures thereof, or the pharmaceutically acceptable salts thereof.

In another embodiment of the present invention, the hydroxamate MMP inhibitors is:

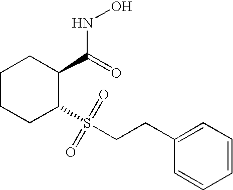

((1S,2R)—N-hydroxy-2-[(2-phenylethyl)sulfonyl]cyclohexanecarboxamide), the optically pure compound, enantiomeric mixtures thereof, or the pharmaceutically acceptable salts thereof.

In another embodiment of the present invention, the hydroxamate MMP inhibitors is:

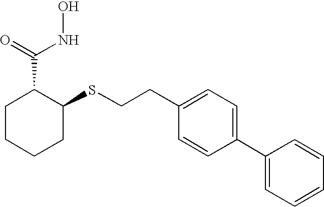

((1S,2R)-2-[(2-biphenyl-4-ylethyl)sulfonyl]-N-hydroxycyclohexanecarboxamide), the optically pure compound, enantiomeric mixtures thereof, or the pharmaceutically acceptable salts thereof.

In another embodiment of the present invention, the hydroxamate MMP inhibitors is:

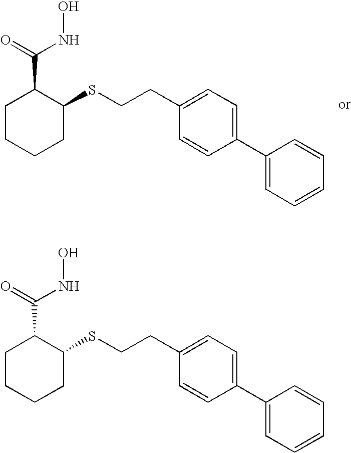

(cis-2-(Biphenyl-4-yl-ethanesulfonyl)-cyclohexanecarboxylic Acid Hydroxamate), the optically pure compound, enantiomeric mixtures thereof, or the pharmaceutically acceptable salts thereof.

In another embodiment of the present invention, the hydroxamate MMP inhibitors is:

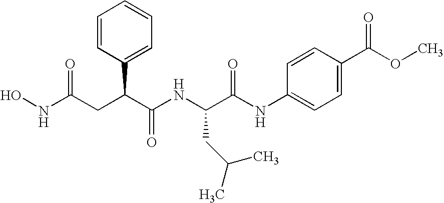

(methyl 4-({N-[(2S)-4-(hydroxyamino)-4-oxo-2-phenylbutanoyl]-L-leucyl}amino)benzoate), the optically pure compound, enantiomeric mixtures thereof, or the pharmaceutically acceptable salts thereof.

In another embodiment of the present invention, the hydroxamate MMP inhibitors is:

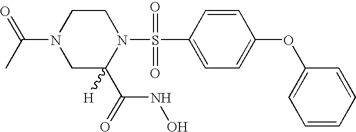

(4-acetyl-N-hydroxy-1-[(4-phenoxyphenyl)sulfonyl]piperazine-2-carboxamide), the optically pure compound, enantiomeric mixtures thereof, or the pharmaceutically acceptable salts thereof.

In another embodiment of the present invention, the hydroxamate MMP inhibitors is:

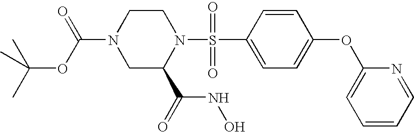

(tert-butyl (3R)-3-[(hydroxyamino)carbonyl]-4-{[4-(pyridin-2-yloxy)phenyl]sulfonyl}piperazine-1-carboxylate), the optically pure compound, enantiomeric mixtures thereof, or the pharmaceutically acceptable salts thereof.

In another embodiment of the present invention, the hydroxamate MMP inhibitors is:

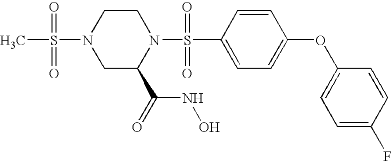

((2R)-1-{[4-(4-fluorophenoxy)phenyl]sulfonyl}-N-hydroxy-4-(methylsulfonyl)piperazine-2-carboxamide), the optically pure compound, enantiomeric mixtures thereof, or the pharmaceutically acceptable salts thereof.

In another embodiment of the present invention, the hydroxamate MMP inhibitors is:

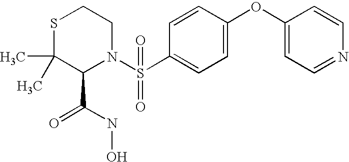

((3S)—N-hydroxy-2,2-dimethyl-4-{[4-(pyridin-4-yloxy)phenyl]sulfonyl}thiomorpholine-3-carboxamide), the optically pure compound, enantiomeric mixtures thereof, or the pharmaceutically acceptable salts thereof.

In another embodiment of the present invention, the hydroxamate MMP inhibitors is:

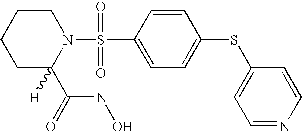

(N-hydroxy-1-{[4-(pyridin-4-ylthio)phenyl]sulfonyl}piperidine-2-carboxamide), the optically pure compound, enantiomeric mixtures thereof, or the pharmaceutically acceptable salts thereof.

In another embodiment of the present invention, the hydroxamate MMP inhibitors is:

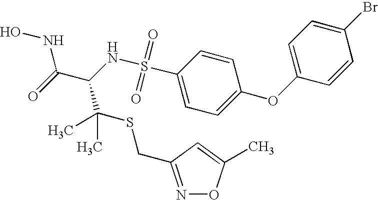

(N²-{[4-(4-bromophenoxy)phenyl]sulfonyl}N¹-hydroxy-3-{[(5-methylisoxazol-3-yl)methyl]thio}-D-valinamide), the optically pure compound, enantiomeric mixtures thereof, or the pharmaceutically acceptable salts thereof.

### DETAILED DESCRIPTION OF THE INVENTION AND PREFERRED EMBODIMENTS

For purposes of the present invention, as described and claimed herein, the following terms are defined as follows:

As used herein, the terms “comprising” and “including” are used in their open, non-limiting sense.

As used herein, the term “alkyl” represents a straight- or branched-chain saturated hydrocarbon, containing 1 to 10 carbon atoms, which may be unsubstituted or substituted by one or more suitable substituents. Exemplary alkyls include, but are not limited to methyl (Me), ethyl (Et), propyl, isopropyl, butyl, isobutyl, t-butyl, and the like.

The term “heteroalkyl” refers to a straight- or branched-chain alkyl group having from 2 to 12 atoms in the chain, one or more of which is a heteroatom selected from S, O, and N. Exemplary heteroalkyls include alkyl ethers, secondary and tertiary amines, alkyl sulfides and the like, which may be unsubstituted or substituted by one or more suitable substituents.

The term “alkenyl” represents a straight- or branched-chain hydrocarbon, containing one or more carbon-carbon double bonds and having 2 to 10 carbon atoms which may be unsubstituted or substituted by one or more suitable substituents. Exemplary alkenyl substituents include, but are not limited to ethenyl, propenyl, butenyl, allyl, pentenyl and the like.

The term “alkynyl”, as used herein, unless otherwise indicated, includes alkyl moieties having at least one carbon-carbon triple bond wherein alkyl is as defined above.

The term “carbocycle” refers to a saturated, partially saturated, unsaturated, or aromatic, monocyclic or fused or non-fused polycyclic, ring structure having only carbon ring atoms (no heteroatoms, i.e., non-carbon ring atoms), which is optionally substituted with a suitable substituent. Exemplary carbocycles include cycloalkyl, aryl, and cycloalkyl-aryl groups.

The term “heterocycle” refers to a saturated, partially saturated, unsaturated, or aromatic, monocyclic or fused or non-fused polycyclic, ring structure having one or more heteroatoms selected from N, O, and S, which is optionally substituted with one or more suitable substituents. Exemplary heterocycles include heterocycloalkyl, heteroaryl, and heterocycloalkyl-heteroaryl groups.

A “cycloalkyl group” is intended to mean a saturated or partially saturated, monocyclic, or fused or spiro polycyclic, ring structure having a total of from 3 to 18 carbon ring atoms (but no heteroatoms), which is optionally substituted with one or more suitable substituents. Exemplary cycloalkyls include cyclopropyl, cyclobutyl, cyclopentyl, cyclopentenyl, cyclohexyl, cycloheptyl, adamantyl, and like groups.

A “heterocycloalkyl group” is intended to mean a monocyclic, or fused or spiro polycyclic, ring structure that is saturated or partially saturated, and has a total of from 3 to 18 ring atoms, including 1 to 5 heteroatoms selected from nitrogen, oxygen, and sulfur, which is optionally substituted with one or more suitable substituents. Illustrative examples of heterocycloalkyl groups include, but are not limited to, pyrrolidinyl, tetrahydrofuryl, piperidinyl, piperazinyl, morpholinyl, thiomorpholinyl, aziridinyl, and like groups.

The term “aryl”, as used herein, unless otherwise indicated, includes an organic radical derived from an aromatic hydrocarbon by removal of one hydrogen, such as phenyl or naphthyl, which is optionally substituted with one or more suitable substituents.

The term “4-10 membered heterocyclic”, as used herein, unless otherwise indicated, includes aromatic and non-aromatic heterocyclic groups containing one to four heteroatoms each selected from O, S and N, wherein each heterocyclic group has from 4-10 atoms in its ring system, and with the proviso that the ring of said group does not contain two adjacent O or S atoms, and which is optionally substituted with one or more suitable substituents. Non-aromatic heterocyclic groups include groups having only 4 atoms in their ring system, but aromatic heterocyclic groups must have at least 5 atoms in their ring system. The heterocyclic groups include benzo-fused ring systems. An example of a 4 membered heterocyclic group is azetidinyl (derived from azetidine). An example of a 5 membered heterocyclic group is thiazolyl and an example of a 10 membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached). An example of a heterocyclic group wherein 2 ring carbon atoms are substituted with oxo (═O) moieties is 1,1-dioxo-thiomorpholinyl.

A “heteroaryl group” is intended to mean a monocyclic or fused or spiro polycyclic, aromatic ring structure having from 4 to 18 ring atoms, including from 1 to 5 heteroatoms selected from nitrogen, oxygen, and sulfur, which is optionally substituted with one or more suitable substituents. Illustrative Examples of heteroaryl groups include pyrrolyl, thienyl, oxazolyl, pyrazolyl, thiazolyl, furyl, pyridinyl, pyrazinyl, triazolyl, tetrazolyl, indolyl, quinolinyl, quinoxalinyl, benzthiazolyl, benzodioxinyl, benzodioxolyl, benzooxazolyl, and the like.

The term “alkoxy”, as used herein, unless otherwise indicated, includes O-alkyl groups wherein alkyl is as defined above.

The term “amino” is intended to mean the —NH₂ radical.

The term “halogen” represents chlorine, fluorine, bromine or iodine.

The term “halo”, as used herein, unless otherwise indicated, means fluoro, chloro, bromo or iodo. Preferred halo groups are fluoro, chloro and bromo.

“A suitable substituent” is intended to mean one or two chemically and pharmaceutically acceptable functional groups, i.e., one or two moieties that do not negate the inhibitory activity of the inventive compounds. Such suitable substituents may be routinely selected by those skilled in the art. Illustrative examples of suitable substituents include, but are not limited to, alkyl groups, hydroxy groups, oxo groups, mercapto groups, alkylthio groups, alkoxy groups, aryl or heteroaryl groups, aralkyl or heteroaralkyl groups, aralkoxy or heteroaralkoxy groups, carboxy groups, amino groups, alkyl- and dialkylamino groups, carbamoyl groups, alkylcarbonyl groups, alkoxycarbonyl groups, alkylaminocarbonyl groups, dialkylaminocarbonyl groups, arylcarbonyl groups, aryloxycarbonyl groups, alkylsulfonyl groups, an arylsulfonyl groups and the like.

The term “a pharmaceutically acceptable salt” refers to a salt that retains the biological effectiveness of the free acids and bases of the specified compound and that is not biologically or otherwise undesirable. A compound of the invention may possess a sufficiently acidic, a sufficiently basic, or both functional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycollates, tartrates, methane-sulfonates, propanesulfonates, naphthalene-1-sulfonates, naphthalene-2-sulfonates, and mandelates.

The term “substituted” means that the specified group or moiety bears one or more substituents. The term “unsubstituted” means that the specified group bears no substituents. The term “optionally substituted” means that the specified group is unsubstituted or substituted by one or more substituents.

The term “HCV-inhibiting agent” means any hydroxamate MMP inhibitor or hydroxamate compound represented by formula I or a pharmaceutically acceptable salt, hydrate, prodrug, active metabolite or solvate thereof.

The term “hydroxamate MMP inhibitor” refers to any MMP inhibitor containing a “—NH—OH”. Examples of hydroxamate MMP inhibitors can be found in, but not limited to, PCT Publication No. WO 00/04892 to Bocan; U.S. Pat. No. 5,985,900 to Bender et. al., U.S. Pat. No. 5753,653 to Bender et. al., and U.S. Pat. No. 6,153,757 to Zook et al., each of which is incorporated herein in their entirety by reference. The hydroxamate MMP inhibitors of the present invention are excellent inhibitors of polymerases, particularly HCV NS5B polymerases. Accordingly, these compounds are capable of targeting and inhibiting HCV polymerases and processes mediated by HCV polymerases. As such, these compounds interfere with the life cycle of viruses, including HCV, and are thus useful as antiviral agents. Inhibition can be measured by various methods as described herein.

The hydroxamate MMP inhibitors of the present invention can be used alone or in combination with HCV immunomodulatory agents, such as alpha-, beta- or gamma-interferons, e.g., Intron A, Roferon A, Infergen, PEG-Intron, Pegasys, Rebetron, Albuferon; other antiviral agents, such as ribavirin (nucleoside analogue), amantadine and merimepodib (VX-497) (and combinations thereof), e.g., PEG-Intron/Rebetol, Pegasys/Copegus, Rebetol/Copegus, Pegasys/CellCept, Pegasys/Amantadine, Pegasys/Amantadine/Ribavirin; immuno-modulating substances, e.g., HCV-AB68; ribozymes, e.g., LY-466700; other inhibitors of HCV polymerase, e.g., MN107/MN283 (prodrug), BC2125; and inhibitors of other targets in the HCV life cycle, including the helicase, e.g., ribavirin-TP; the protease, e.g., BILN-2061, SCH-6 (ketoamide), VX-950; the metalloprotease or the internal ribosome entry site, e.g., ISIS-14803 (antisense); or combinations of thereof.

The term “hydroxamate compound” refers to any compounds containing a “—NH—OH”.

The term “processes mediated by HCV polymerase”, as used herein, refers to biological, physiological, endocrinological, and other bodily processes which are mediated by receptor or receptor combinations which are responsive to the hydroxamate MMP inhibitors described herein (e.g., hepatitis C or chronic liver disease, including cirrhosis and hepatocellular carcinoma (Hoofnagle, J. H.; 1997; Hepatology 26: 15S-20S, incorporated herein by reference), the formation of macrophages which lead to the development of atherosclerotic plaques, and the like). Modulation of such processes can be accomplished in vitro or in vivo. In vivo modulation can be carried out in a wide range of subjects, such as, for example, humans, rodents, sheep, pigs, cows, and the like.

The term “interfering with, decreasing or preventing” HCV viral replication in a cell means to reduce HCV replication or production of HCV components necessary for progeny virus in a cell as compared to a cell not being transiently or stably transduced with the ribozyme or a vector encoding the ribozyme. Simple and convenient assays to determine if HCV viral replication has been reduced include an ELISA assay for the presence, absence, or reduced presence of anti-HCV antibodies in the blood of the subject (Nasoff et al., PNAS 88:5462-5466, 1991), RT-PCR (Yu et al., in Viral Hepatitis and Liver Disease 574-477, Nishioka, Suzuki and Mishiro (Eds.); Springer-Verlag Tokyo, 1994) or liver function tests. Such methods are well known to those of ordinary skill in the art. Alternatively, total RNA from transduced and infected “control” cells can be isolated and subjected to analysis by dot blot or northern blot and probed with HCV specific DNA to determine if HCV replication is reduced. Alternatively, reduction of HCV protein expression can also be used as an indicator of inhibition of HCV replication. A greater than fifty percent reduction in HCV replication as compared to control cells typically quantitates a prevention of HCV replication.

The term “pharmaceutically acceptable carries” refers to a carrier or adjuvant that may be administered to a patient, together with a compound of this invention, and which does not destroy the pharmacological activity thereof and is nontoxic when administered in doses sufficient to deliver a therapeutic amount of the compound.

The term “prodrug” is a compound that may be converted under physiological conditions or by solvolysis to the specified compound or to a pharmaceutically acceptable salt of such compound. A prodrug may be a derivative of one of the hydroxamate compounds of the present invention that contains a moiety, such as for example —CO₂R,—PO(OR)₂ or —C═NR, that may be cleaved under physiological conditions or by solvolysis. Any suitable R substituent may be used that provides a pharmaceutically acceptable solvolysis or cleavage product. A prodrug containing such a moiety may be prepared according to conventional procedures by treatment of a hydroxamate compound of this invention containing, for example, an amido, carboxylic acid, or hydroxyl moiety with a suitable reagent.

The term “active metabolite” refers to a pharmacologically active product produced through metabolism in the body of a specified hydroxamate compound or salt thereof.

Prodrugs and active metabolites of the hydroxamate compound may be identified using routine techniques known in the art. See, e.g., Bertolini et al., J. Med. Chem., 40:2011-2016 (1997); Shan et al., J. Pharm. Sci., 86 (7):765-767 (1997); Bagshawe, Drug Dev. Res., 34:220-230 (1995); Bodor, Advances in Drug Res., 13:224-331 (1984); Bundgaard, “Design of Prodrugs” (Elsevier Press, 1985); Larsen, Design and Application of Prodrugs, Drug Design and Development (Krogsgaard-Larsen et al. eds., Harwood Academic Publishers, 1991); Dear et al., Chromatogr. B, 748:281-293 (2000); Spraul et al., J. Pharmaceutical & Biomedical Analysis, 10 (8):601-605 (1992); and Prox et al., Xenobiol, 3(2):103-112 (1992).

The term “solvate” is intended to mean a pharmaceutically acceptable solvate form of a specified compound that retains the biological effectiveness of such compound. Examples of solvates include compounds of the invention in combination with water, isopropanol, ethanol, methanol, DMSO, ethyl acetate, acetic acid, or ethanolamine.

If a hydroxamate compound used in the method of the invention is a base, a desired salt may be prepared by any suitable method known to the art, including treatment of the free base with an inorganic acid (such as hydrochloric acid, hydrobromic acid, sulfuric acid, nitric acid, phosphoric acid, and the like), or with an organic acid (such as acetic acid, maleic acid, succinic acid, mandelic acid, fumaric acid, malonic acid, pyruvic acid, oxalic acid, glycolic acid, salicylic acid, pyranosidyl acid (such as glucuronic acid or galacturonic acid), alpha-hydroxy acid (such as citric acid or tartaric acid), amino acid (such as aspartic acid or glutamic acid), aromatic acid (such as benzoic acid or cinnamic acid), sulfonic acid (such as p-toluenesulfonic acid or ethanesulfonic acid), and the like.

If a hydroxamate compound used in the method of the invention is an acid, a desired salt may be prepared by any suitable method known to the art, including treatment of the free acid with an inorganic or organic base (such as an amine (primary, secondary, or tertiary)), an alkali metal hydroxide, or alkaline earth metal hydroxide. Illustrative examples of suitable salts include organic salts derived from amino acids (such as glycine and arginine), ammonia, primary amines, secondary amines, tertiary amines, and cyclic amines (such as piperidine, morpholine, and piperazine), as well as inorganic salts derived from sodium, calcium, potassium, magnesium, manganese, iron, copper, zinc, aluminum, and lithium.

In the case of hydroxamate compound, prodrugs, salts, or solvates that are solids, it is understood by those skilled in the art that the hydroxamate compound, prodrugs, salts, and solvates used in the method of the invention, may exist in different polymorph or crystal forms, all of which are intended to be within the scope of the present invention and specified formulas. In addition, the hydroxamate compound, salts, prodrugs and solvates used in the method of the invention may exist as tautomers, all of which are intended to be within the broad scope of the present invention.

In some cases, the hydroxamate compound, salts, prodrugs and solvates used in the method of the invention may have chiral centers. When chiral centers are present, the hydroxamate compound, salts, prodrugs and solvates may exist as single stereoisomers, racemates, and/or mixtures of enantiomers and/or diastereomers. All such single stereoisomers, racemates, and mixtures thereof are intended to be within the broad scope of the present invention.

The compounds of the present invention may have asymmetric carbon atoms. The carbon-carbon bonds in the compounds of the present invention may be depicted herein using a solid line ( [symbol] ), a solid wedge ( [symbol] ), or a dotted wedge ( [symbol] ) The use of a solid line to depict bonds to asymmetric carbon atoms is meant to indicate that all possible stereoisomers at that carbon atom are included. The use of either a solid or dotted wedge to depict bonds to asymmetric carbon atoms is meant to indicate that only the stereoisomer shown is meant to be included. It is possible that compounds of the invention may contain more than one asymmetric carbon atom. In those compounds, the use of a solid line to depict bonds to asymmetric carbon atoms is meant to indicate that all possible stereoisomers are meant to be included. The use of a solid line to depict bonds to one or more asymmetric carbon atoms in a compound of the invention and the use of a solid or dotted wedge to depict bonds to other asymmetric carbon atoms in the same compound is meant to indicate that a mixture of diastereomers is present.

When used describe a particular compound, the term “optically active” is used herein to indicate that the compound is enantiomerically or diastereomerically enriched. Compounds that are enantiomerically enriched contain greater than 50% of a single stereoisomer, and preferably contain greater than 75% of a single stereoisomer. Compounds that are diastereomerically enriched contain greater than 50% of a single stereoisomer of each chiral carbon center present in the diastereomer, and preferably contain greater than 75% of a single stereoisomer of each chiral carbon present in the diastereomer. Preferably, however, the compounds are present in optically pure form.

When used describe a particular compound, the term “optically pure” is used herein to indicate that the compound is substantially enantiomerically or diastereomerically pure. Compounds that are substantially enatiomerically pure contain at least 90% of a single isomer and preferably contain at least 95% of a single isomer. Compounds that are substantially diastereomerically pure contain at least 90% of a single isomer of each chiral carbon center present in the diastereomer, and preferably contain at least 95% of a single isomer of each chiral carbon. More preferably, the optically pure compounds in this invention contain at least 97.5% of a single isomer and most preferably contain at least 99% of a single isomer. Compounds identified herein as single stereoisomers are meant to describe compounds that are present in a form that contains at least 90% of a single isomer.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

The activity of the hydroxamate compound as inhibitors of HCV activity may be measured by any of the suitable methods available in the art, including in vivo and in vitro assays. An Example of a suitable assay for activity measurements is the HCV replicon assay described herein.

Administration of the hydroxamate compound and their pharmaceutically acceptable prodrugs, salts, active metabolites, and solvates may be performed according to any of the accepted modes of administration available to those skilled in the art. Illustrative Examples of suitable modes of administration include oral, nasal, parenteral, topical, transdermal, and rectal. Oral and intravenous deliveries are preferred.

An HCV-inhibiting agent may be administered as a pharmaceutical composition in any suitable pharmaceutical form. Suitable pharmaceutical forms include solid, semisolid, liquid, or lyopholized formulations, such as tablets, powders, capsules, suppositories, suspensions, liposomes, and aerosols. The HCV-inhibiting agent may be prepared as a solution using any of a variety of methodologies. For Example, the HCV-inhibiting agent can be dissolved with acid (e.g., 1 M HCl) and diluted with a sufficient volume of a solution of 5% dextrose in water (D5W) to yield the desired final concentration of HCV-inhibiting agent (e.g., about 15 mM). Alternatively, a solution of D5W containing about 15 mM HCl can be used to provide a solution of the HCV-inhibiting agent at the appropriate concentration. Further, the HCV-inhibiting agent can be prepared as a suspension using, for example, a 1% solution of carboxymethylcellulose (CMC).

Acceptable methods of preparing suitable pharmaceutical forms of the pharmaceutical compositions are known or may be routinely determined by those skilled in the art. For Example, pharmaceutical preparations may be prepared following conventional techniques of the pharmaceutical chemist involving steps such as mixing, granulating, and compressing when necessary for tablet forms, or mixing, filling, and dissolving the ingredients as appropriate, to give the desired products for oral, parenteral, topical, intravaginal, intranasal, intrabronchial, intraocular, intraaural, and/or rectal administration.

Pharmaceutical compositions of the invention may also include suitable excipients, diluents, vehicles, and carriers, as well as other pharmaceutically active agents, depending upon the intended use. Solid or liquid pharmaceutically acceptable carriers, diluents, vehicles, or excipients may be employed in the pharmaceutical compositions. Illustrative solid carriers include starch, lactose, calcium sulfate dihydrate, terra alba, sucrose, talc, gelatin, pectin, acacia, magnesium stearate, and stearic acid. Illustrative liquid carriers include syrup, peanut oil, olive oil, saline solution, and water. The carrier or diluent may include a suitable prolonged-release material, such as glyceryl monostearate or glyceryl distearate, alone or with a wax. When a liquid carrier is used, the preparation may be in the form of a syrup, elixir, emulsion, soft gelatin capsule, sterile injectable liquid (e.g., solution), or a nonaqueous or aqueous liquid suspension.

A dose of the pharmaceutical composition may contain at least a therapeutically effective amount of an HCV-inhibiting agent and preferably is made up of one or more pharmaceutical dosage units. The selected dose may be administered to a mammal, for example, a human patient, in need of treatment mediated by inhibition of HCV activity, by any known or suitable method of administering the dose, including topically, for example, as an ointment or cream; orally; rectally, for example, as a suppository; parenterally by injection; intravenously; or continuously by intravaginal, intranasal, intrabronchial, intraaural, or intraocular infusion. When the composition is administered in conjunction with a cytotoxic drug, the composition can be administered before, with, and/or after introduction of the cytotoxic drug. However, when the composition is administered in conjunction with radiotherapy, the composition is preferably introduced before radiotherapy is commenced.

The phrases “therapeutically effective amount” and “effective amount” are intended to mean the amount of an inventive agent that, when administered to a mammal in need of treatment, is sufficient to effect treatment for injury or disease conditions alleviated by the inhibition of HCV viral replication. The amount of a given HCV-inihibiting agent used in the method of the invention that will be therapeutically effective will vary depending upon factors such as the particular HCV-inihibiting agent, the disease condition and the severity thereof, the identity and characteristics of the mammal in need thereof, which amount may be routinely determined by artisans.

It will be appreciated that the actual dosages of the HCV-inhibiting agents used in the pharmaceutical compositions of this invention will be selected according to the properties of the particular agent being used, the particular composition formulated, the mode of administration and the particular site, and the host and condition being treated. Optimal dosages for a given set of conditions can be ascertained by those skilled in the art using conventional dosage-determination tests. For oral administration, e.g., a dose that may be employed is from about 0.001 to about 1000 mg/kg body weight, preferably from about 0.1 to about 100 mg/kg body weight, and even more preferably from about 1 to about 50 mg/kg body weight, with courses of treatment repeated at appropriate intervals.

By using the hydroxamate MMP inhibitors to decrease or prevent HCV viral replication activity as described herein, one can further identify cellular or viral pathways interfering with the functioning of HCV polymerase which could be used for treating indications caused by HCV infections, e.g., by administering a therapeutically effective amount of an MMP inhibitor to a patient in need thereof. See, e.g., Love et al., J Virol. 2003 Jul;77(13):7575-81.

“Gene profiling experiments,” as the term is used herein, may be performed by methods known in the art. For example, microarray analyses of RNA from hepatitis C virus (HCV)-infected cirrhotic livers can be performed to identify a gene expression signature of liver disease. The expression levels of genes can be analyzed using surgical material and core biopsy specimens from HCV-infected cirrhotic liver explants in comparison with reference samples of normal nondiseased liver. In addition, normal liver samples can be compared with each other to determine normal physiologic variation in gene expression. A set of genes, including some associated with stress, acute-phase immune response, and hepatic stellate cell activation, may have variable expression levels in normal livers. These genes can be subtracted from the sets of genes differentially expressed in cirrhotic livers. To exclude cancer-related genes from marker sets, one can subtract genes that also expressed differentially in hepatocellular carcinomas. The resultant HCV- and liver disease-associated gene set can provide a molecular portrait of several processes occurring in the HCV-infected liver. The gene set may include (1) genes expressed in activated lymphocytes infiltrating the cirrhotic liver, and activated liver macrophages; (2) genes involved in remodeling of extracellular matrix-cell and cell-cell interactions associated with cytoskeleton rearrangements; (3) genes related to an anti-apoptotic signaling pathway; and (4) genes involved with interferon response and virus-host interactions. Using said microarray analysis, one can identify potential gene markers of HCV-associated liver disease, and such markers can be used to generate a database of experiments describing HCV pathogenesis. See, e.g., Shackel et al., Am J Pathol. 2002 Feb;160(2):641-54; Kato et al., Hepatology, 2000 Aug;32(2):405-12.

### EXAMPLES

In the examples described below, unless otherwise indicated, all temperatures are set forth in degrees Celsius and all parts and percentages are by weight. Reagents were purchased from commercial suppliers, such as Sigma-Aldrich Chemical Company, or Lancaster Synthesis Ltd. and were used without further purification unless otherwise indicated. Tetrahydrofuran (THF) and N, N-dimethylformamide (DMF) were purchased from Aldrich in Sure Seal bottles and used as received. All solvents were purified using standard methods known to those skilled in the art, unless otherwise indicated.

The reactions set forth below were done generally under a positive pressure of argon at an ambient temperature (unless otherwise stated) in anhydrous solvents, and the reaction flasks were fitted with rubber septa for the introduction of substrates and reagents via syringe. Glassware was oven dried and/or heat dried. Analytical thin layer chromatography (TLC) was performed on glass-backed silica gel 60 F 254 plates from Analtech (0.25 mm), eluted with the appropriate solvent ratios (v/v), and are denoted where appropriate. The reactions were assayed by TLC, HPLC, or ¹H NMR, and terminated as judged by the consumption of starting material.

Visualization of the TLC plates was done with iodine vapor, ultraviolet illumination, 2% Ce(NH₄)₄(SO₄)₄ in 20% aqueous sulfuric acid, 2% ninhydrin in ethanol, or p-anisaldehyde spray reagent, and activated with heat where appropriate. Work-ups were typically done by doubling the reaction volume with the reaction solvent or extraction solvent and then washing with the indicated aqueous solutions using 25% by volume of the extraction volume unless otherwise indicated. Product solutions were dried over anhydrous Na₂SO₄ and/or MgSO₄ prior to filtration and evaporation of the solvents under reduced pressure on a rotary evaporator and noted as solvents removed in vacuo. Flash column chromatography (Still et al., J. Org. Chem., 1978, 43, 2923-2924) was done using Merck silica gel (47-61 μm) with a silica gel crude material ratio of about 20:1 to 50:1, unless otherwise stated. Certain example compounds were purified via preparative high-performance liquid chromatography (HPLC), and unless otherwise indicated, refers to a Gilson 321 system, equipped with a C18 reversed-phase preparative column (Metasil AQ 10 micron, 120A, 250×21.2 mm, MetaChem) and elution with a gradient of 0.1% trifluoroacetic acid (TFA)/5% acetonitrile/water to 0.1% TFA/5% water/acetonitrile over 20 min and flow rate of 20 mUmin. Hydrogenations were performed at ambient pressure unless otherwise indicated. All melting points (mp) are uncorrected.

¹H-NMR spectra were recorded on a Bruker or Varian instrument operating at 300 MHz and ¹³C-NMR spectra were recorded operating at 75 MHz. NMR spectra were obtained as CDCl₃ solutions (reported in ppm), using chloroform as the reference standard (7.27 ppm and 77.00 ppm) unless otherwise indicated. When peak multiplicities are reported, the following abbreviations are used: s (singlet), d (doublet), t (triplet), q (quartet), m (multiplet), bs (broad singlet), bm (broad multiplet), dd (doublet of doublets), ddd (doublet of doublet of doublets), dddd (doublet of doublet of doublet of doublets), dt (doublet of triplets). Coupling constants, when given, are reported in Hertz (Hz).

Infrared (IR) spectra were recorded on a Perkin-Elmer FT-IR Spectrometer as neat oils, KBr pellets, or CDCl₃ solutions, and when given are reported in wave numbers (cm⁻¹). Mass spectrometry was conducted with various techniques. Matrix-Assisted Laser Desorption/Ionization Fourier Transform Mass Spectrometry (MALDI FTMS), was performed on an lonSpec FTMS mass spectrometer. Samples are irradiated with a nitrogen laser (Laser Science Inc.) operated at 337 nm and the laser beam is attenuated by a variable attenuator and focused on the sample target. The ions are then differentiated according to their m/z using an ion cyclotron resonance mass analyzer. The electrospray ionization (ESI) mass spectrometry experiments were performed on an API 100 Perkin Elmer SCIEX single quadrupole mass spectrometer. Electrospray samples are typically introduced into the mass analyzer at a rate of 4.0 μl/minute. The positive and negative ions, generated by charged droplet evaporation, enter the analyzer through an interface plate and a 100 mm orifice, while the declustering potential is maintained between 50 and 200V to control the collisional energy of the ions entering the mass analyzer. The emitter voltage is typically maintained at 4000V. The liquid chromatography (LC) electrospray ionization (ESI) mass spectrometry experiments were performed on an Hewlett-Packard (HP) 1100 MSD single quadrupole mass spectrometer. Electrospray samples are typically introduced into the mass analyzer at a rate of 100 to 1000 μl/minute. The positive and negative ions, generated by charged droplet evaporation, enter the analyzer through a heated capillary plate, while the declustering potential is maintained between 100 and 300V to control the collisional energy of the ions entering the mass analyzer. The emitter voltage is typically maintained at 4000V.

Hydroxamate MMP inhibitors as used in the method of the present invention can be prepared as described in lPCT Publication No. WO 00/04892 to Bocan; U.S. Pat. No. 5,985,900 to Bender et. al., and U.S. Pat. No. 5753,653 to Bender et. al., each of which is incorporated herein in their entirety by reference.

Preferred compounds in accordance with the invention may be prepared in manners analogous to those specifically described below.

### Example 1

### 2-(2-Biphenyl-4-yl-ethylsulfanyl)-cyclohex-1-ene-carboxylic Acid Hydroxyamide or 2-[(2-biphenyl-4-ylethyl)thio]-N-hydroxycyclohex-1-ene-1-carboxamide

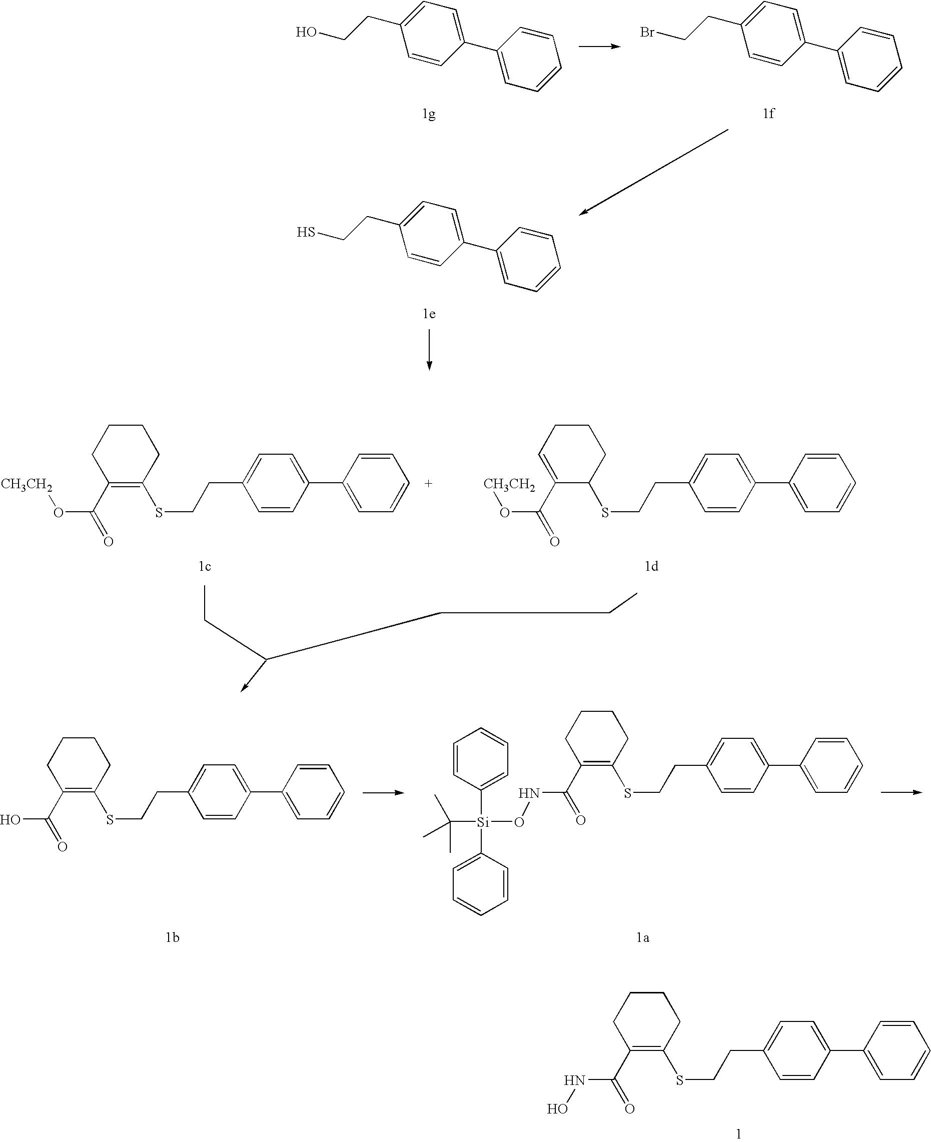

A solution of compound 1a (0.066 g, 0.11 mmol), 1N N,N,N,N-tetrabutylammonium fluoride (TBAF) in tetrahydrofuran (THF; 22 mL, 22 mmol) and THF (I mL) stirred at ambient temperature for 25 minutes. To the solution was added ethyl acetate (30 mL), then the solution was washed with H₂O (3×20 mL), brine (20 mL), dried, and evaporated to give an oil, 0.065 g. The crude product was purified by column chromatography (stepwise gradient 20% ethyl acetate/hexane-100%/ethyl acetate) and crystallized from some fractions to give a white solid (4 mg, 10% yield). ¹H NMR (CDC1₃) 7.59-7.14 (9H, m), 3.07-2.83 (4H, m), 2.36(1H, m), 1.65-1.50 (7H, m). HRFABMS Calcd for C₂₁H₂₃O₂SNa: 376.1347. Found 376.1358.

### Preparation of Compound 1a: 2-(2-Biphenyl-4-yl-ethylsulfanyl)-cyclohex-1-ene-carboxylic Acid (O-tert-Butyidiphenylsilyl) Hydroxyamide

A solution of compound 1b (247 mg, 0.730 mmol), O-dimethyl-tert-butylsilyl hydroxylamine (299 mg, 1.10 mmol, 1.5 eq), and 1-[3-(dimethylamino)-propyl]-3-ethylcarbodiimide hydrochloride (EDC; 280 mg, 1.46 mmol, 2.00 eq) in CH₂Cl₂ (4 mL) stirred at ambient temperature for 18 hours. Added CH₂Cl₂ (30 mL), washed with H₂O (40 mL), dried, and evaporated to give a crude product (0.3 g), which was purified by column chromatography (CH₂Cl₂) to give 0.19 g (44%) of a solid, which was used without further purification. ¹H NMR (CDCl₃) δ 8.45 (1H, bs), 8.75-7.15 (20H, m), 2.90 (2H, m), 2.65 (2H, m), 2.15 (2H, m), 1.45 (4H, m), 1.10 (2H, m).

### Preparation of Compound 1b: 2-(2-Biphenyl-4-yl-ethylsulfanyl)-cyclohex-1-ene-carboxylic Acid

The crude mixture of compounds 1c and 1d (1.81 g; 4.94 mmol), 1N KOH (20 mL, 4 eq), and ethanol (15 ml) was heated at reflux for 5 hours, allowed to cool, and evaporated. The resultant residue was treated with water (30 mL), washed with ethyl acetate (3×30 ml), acidified with 6N HCl, and extracted with ethyl acetate (2×30 mL). The acidified, latter organic extracts were washed with brine (30 ml) and concentrated in vacuo. The specific titular isomer was isolated by crystallization from ethanol/hexanes. The mother liquor also provided the other possible isomer 6-(2-biphenyl-4-yl-ethylsulfanyl)-cyclohex-1-ene-carboxylic acid, see Example 2 below. ¹H NMR (DMSO-d₆): δ 12.14 (1H, bs), 7.67-7.60 (9H, m), 4.10 (2H, s), 2.54 (2H, s), 2.24 (2H, s), 1.60-1.53 (4H, m). Anal. For C₂₀H₂₀OS: C, 74,04, H, 6.21; S, 9.88. Found C, 73,81; H, 6.26; S, 9.78.

### Preparation of Compounds 1c and 1d: 2-(2-Biphenyl-4-yl-ethylsulfanyl)-cyclohex-1-ene-carboxylic Acid Ethyl Ester and 6-(2-Biphenyl-4-yl-ethylsulfanyl)-cyclohex-1ene-carboxylic Acid Ethyl Ester

A mixture of compound 1e (630 mg, 2.94 mmol), 2-oxo-cyclohexane-carboxylic acid ethyl ester (500 mg, 2.94 mmol), and Montmorillonite K10 (0.6 g) in toluene (30 mL) was heated at reflux for 4.5 h. Allowed to cool, filtered, and solvent evaporated to give a light-yellow oil (873 mg, 81%), which was a mixture of isomers by NMR and used without further purification.

### Preparation of Compound 1e: 2-Biphenyl-4-yl-ethane-thiol

A solution of compound 1f (1.5 g, 3.15 mmol) and thiourea (0.62 g, 8.1 mmol, 1.1 eq) in dioxane (15 mL) was heated at reflux for 1 hour. After cooling, the white isothiouronium chloride was filtered off, suspended in 20% NaOH (40 mL) and heated at reflex for 4.5 hours. Allowed to cool, added H₂O (40 mL) and refluxed for an additional 2 hours. The mixture was then filtered, acidified, poured into H₂O (100 mL) and extracted with ethyl acetate (2×50 mL). The organic layers were combined, dried, and evaporated to give a crude solid which was recrystallized from hexane to give white plates (446 mg, 66%), which was used without further purification.

### Preparation of Compound 1f: 4-(2-Bromo-ethyl)-biphenyl (48)

A solution of compound 1g (4.38 g, 22.1 mmol), and CBr₄ (8.79 g, 26.5 mmol) in CH₂Cl₂ (40 mL) was cooled to 0° C., treated with PPh₃ (8.69 g, 33.1 mmol), and stirred for 0.5 h. The solvent was removed, diluted with diethyl ether (100 mL), and filtered. The extract was concentrated and purified by column chromatography (1:1 ethyl acetate/hexane) to give a yellow oil in quantitative yield, which displayed an NMR that matched literature (Kawasaki, M.; Goto, M.; Kawabata, S.; Kometani, T. Tetrahedron: Asymmetry 2001, 12, 585-596) and was used without further purification. ¹H NMR (CDC1₃) δ 7.60-7.26 (9H, m), 3.61 (2H,t, J=7.7 H z), 3.21 (2H, t, J=7.7 Hz).

### Preparation of Compound 1g: 2-Biphenyl-4-yl-ethanol

A solution of 4-biphenylacetic acid (10.61 g, 50.00 mmol, 1 eq) in THF (100 mL) was added dropwise over a 30 min to a slurry of LiAIH₄ (4.74 g, 125 mmol) in THF (80 mL) at 0° C. The resultant mixture was heated at reflux for 1.5 hours, re-cooled, carefully quenched with 6N HCl (200 mL), and extracted with diethyl ether (200 mL). The organic layer was washed with H₂O (300 mL), brine (300 mL), and concentrated to give a solid, which was crystallized from toluene/hexanes to give a cream-colored solid (7.68 g, 78%), which displayed an NMR that matched literature (Kawasaki, M.; Goto, M.; Kawabata, S.; Kometani, T. Tetrahedron: Asymmetry 2001, 12, 585-596) and was used without further purification.

### Example 2

### 6-(2-Biphenyl-4-yl-ethylsulfanyl)-cyclohex-1-ene-carboxylic Acid Hydroxyamide

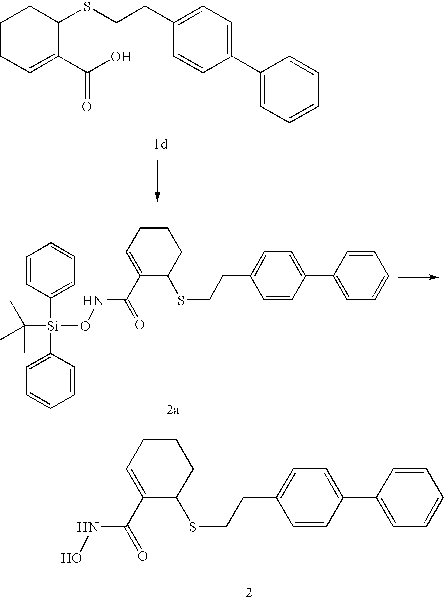

The compound of Example 2 was prepared in the same manner as example 1, from compound 2a, as a cream-colored solid after recrystallization from ethanol/hexanes (28%). mp 166-168° C. ¹H NMR (DMSO-d₆): δ10.51 (1H, s), 8.83 (1H, s), 7.63-7.29 (9H, m), 5.78 (1H, s), 3.88 (2H, s), 3.30 (1H, s), 2.86 (1H, s), 1.95 (2H,bs), 1.70 (3H, m). HRFABMS Calcd for C₂₀H₂₀NO₂S: 340.1371, found 340.1364. Anal Calcd for C₂₀H₂₀NO₂S: C, 70.77; H, 6.24; N, 4.13, S, 9.44. Found C, 70.64; H, 6.24; N, 4.1S; S, 9.54.

### Preparation of Compound 2a: 6-(2-Biphenyl-4-yl-ethylsulfanyl)-cyclohex-1-ene-carboxylic Acid (O-Dimethyl-tert-butylsilyl)-hydroxyamide

This compound was obtained in the same fashion as compound 1a in Example 1, from compound 1b, as a white solid in 8% yield after column chromatography (30% ethyl acetate/hexane): mp 135-137° C. ¹H NMR (DMSO-d₆): δ10.32 (1H, s), 8.77 (1H, s), 7,63-7.29 (9H, m), 3.96 (2H, s), 2.22 (2H, s), 2.13 (2H, s), 1.51 (4H, bs). HRFABMS Calcd for C₂₀H₂₂O₂S: 340.1371, found 340.1365.

Preparation of Compound 1d: 6-(2-Biphenyl-4-yl-ethylsulfanyl)-cyclohex-1-ene-carboxylic Acid

The present compound was isolated upon concentration of the mother liquor from the crystallization of compounds 1c and 1d in Example 1 and purification of the oily residue by column chromatography (ethyl acetate) to give an oil (23% yield). ¹H NMR (DMSO-d₆): δ 12,30 (1H, s), 7.67-7.32 (9H. m), 5.84 (1H, s), 3.96 (2H, s), 1H, bs), 2.00(2H,bs), 1.85 (2H, m), 1.50 (2H, m). Anal. Calcd for C₂₀H₂₀O₂S: C, 74.04; H, 6.21, S, 9.88. Found C, 74. 15; H, 6 77, S, 9.17.1

### Example 3

### cis-Phenethylsulfanyl-cyclohexanecarboxylic Acid Hydroxyamide or (1R,2R)—N-hydroxy-2-[(2-phenylethyl)thio]cyclohexanecarboxamide

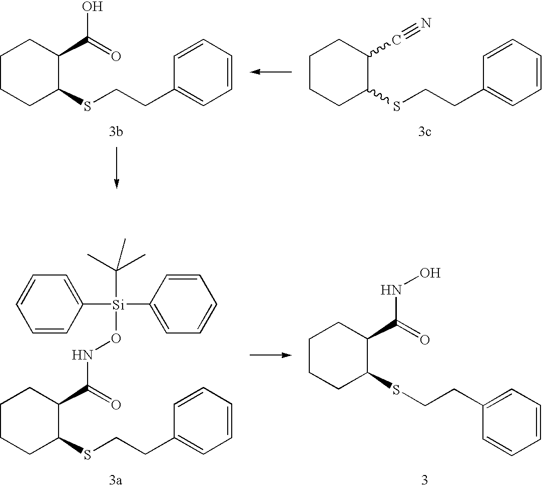

Compound 3 was prepared as described in Example 1, from compound 3a. Recrystallization from diethyl ether/hexanes gave a white solid (0.061 g, 44%).

### Preparation of Compound 3a: cis-Phenethylsulfanyl-cyclohexanecarboxylic Acid (O-Dimethyl-tert-butylsilyl)-hydroxyamide

This compound was prepared as described for compound 1a in Example 1, from compound 3b. Purification was achieved by column chromatography (20% ethyl acetate/hexanes) gave a white solid (0.19 g, 65%), which was used without further purification. ¹H NMR (DMSO-d₆): δ 10.40 (1H, s), 8.71 (1H, s), 7.38-7.26 (5H, m), 3.43 (s, 4H), 3.15 (bs, 1H), 2.86(3H, m), 2.55(1H, m), 2.00 (1H, m), 1.65 (3H, m), 1.30 (1H, m). HRFABMS Calcd for C₁₅H₂₁NO₂SCs: 412.0347, found 412.0367.

### Preparation of Compound 3b: cis-2-Phenethylsulfanyl-cyclohexanecarboxylic Acid

The present compound was obtained by heating a mixture of compound 3c (0.37 g, 1.5 mmol), 2N H₂SO₄ (2 mL), conc. H₂SO₄ (4 mL), and dioxane (20 mL) at reflux for 14 hours. The solvent was evaporated and extracted with diethyl ether (2×30 mL). The combined organic layers were washed with H₂O (20 mL), brine (20 mL) and dried to give an oil (0.28 g) which was purified by column chromatography (70% ethanol/hexanes) to give 0.22 g (55%) of a viscous oil which slowly solidified on standing. ¹H NMR (CDCl₃): δ 7.38-7.20 (5H, m), 3.36 (1H, bs), 2.91-2.74 (4H, m), 1.96(1H, m), 1.71 (6H, m), 1.50 (1H, m), 1.30 (1H, m). Anal. Calcd for C₁₅H₂₀O₂S: C, 68.14; H, 7.62; S. 12.13. Found: C, 68.14, H. 7.66; S. 12.06.

### Preparation of Compound 3c: cis/trans-2-Phenethylsulfanyl-cyclohexane Carbonitrile

A mixture of cyclohex-1-ene carbonitrile (1.61 g, 15.0 mmol), and phenethyl mercaptan (6.0 mL, 45 mmol) in piperidine (30 mL) was combined in a pressure tube, evacuated, and heated at reflux for 6 h. The reaction mixture was then poured into 3N HCl (150 mL) and extracted with ethyl acetate (125 mL). The organic layer was washed with diethyl ether (150 mL), brine (150 mL), dried and concentrated to give a light orange oil, the cis/trans isomers were separated by column chromatography (10% ethyl acetate/hexanes) to give a total yield of 2.2 g (60% total), of which 1.16 g (80%) was the cis isomer. ¹H NMR (CDCl₃): δ 7.33-7.20 (5H, m), 3.07(1H, bs), 2.86 (2H, bm), 2.69 (1H, bm), 2.10(1H, m), 1.90-1.50(6H, m), 1.30 (1H, m). Anal. Calcd for C₁₅H₁₉NS: C, 73,42; H. 7.80; N, 5.70, S. 13.07 Found: C, 73.18; H, 7.80, N, 5.68; S. 13.04. Trans isomer: ¹H NMR (CDCl₃): δ 7.36-7.12 (5H, m), 2.93 (2H, bm), 2.70 (1H, m), 2.54 (1H, m), 2.12 (2H, m), 1.74-1.58 (4H, m), 1.37 (2H, m).

### Example 4

### cis-Phenyl-ethanesulfonyl-cyclohexanecarboxylic Acid Hydroxyamide

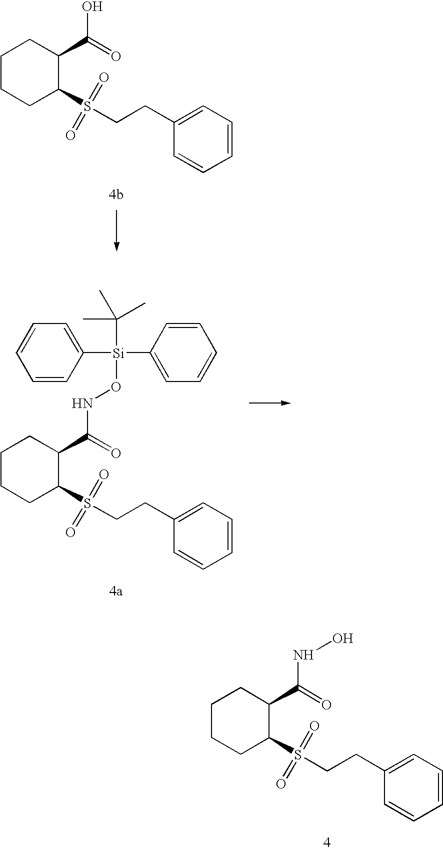

The present compound was prepared as described in Example 1, from compound 4a with a reaction time of 1 hour. Purification was performed by column chromatography (ethyl acetate/trace Acetic acid), which provided a white foamy solid (35%). ¹H NMR (CDCl₃): δ 7.36-7.21 (5H, m), 3.30-2.90 (6H, m), 2.30 (1H, m), 2.10-1.85 (4H, m),1.45(2H, m), 1.20 (1H, m). HRFABMS. Calcd for C₁₅H₂₁NO₄SNa: 334.1089, found 334.1082. Anal. (C₁₅H₂₁NO₄S.0.25 H₂O) C, 57.03; H, 6.86; N, 4.43, S,10.15. Found: C, 57.09; H, 6.87, N, 4.33; S, 10.04.

### Preparation of Compound 4a: cis-Phenylethanesulfonyl-cyclohexanecarboxylic Acid (O-Dimethyl-tert-butylsilyl)-hydroxyamide

The present compound was prepared in the same fashion as compound 1a in Example 1 from compound 4b with a reaction time of 1 hour to give a colorless oil (89%), which was used without any further purification.

### Preparation of Compound 4b: cis-2-Phenylethanesulfonyl-cyclohexanecarboxylic Acid

To a solution of compound 3b (from Example 3); 50 mg, 0.19 mmol) in methanol (1.5 mL) at 0° C. was added a mixture of oxone (0.46 g, 0.76 mmol, 4 eq) in H₂O (1.5 mL) in one portion. The resulting slurry stirred at ambient temperature for 65 h. Diluted with H₂O (10 mL) and extracted with CHCl₃ (3×10 mL). The combined organic layers were dried and concentrated to give colorless oil (0.052 g, 93%), which was used without further purification. ¹H NMR (CDCl₃): δ 7.40-7.20 (5H, m), 3.45-3.10 (5H, m), 2.30 (1H, m), 2.20 (1H, m), 1.95 (2H, m), 1.55 (3H, m), 1.30 (2H, m).

### Example 5

### trans-2-Phenylethanesulfonyl-cyclohexanecarboxylic Acid Hydroxyamide or (1S,2R)—N-hydroxy-2-[(2-phenylethyl)sulfonyl]cyclohexanecarboxamide

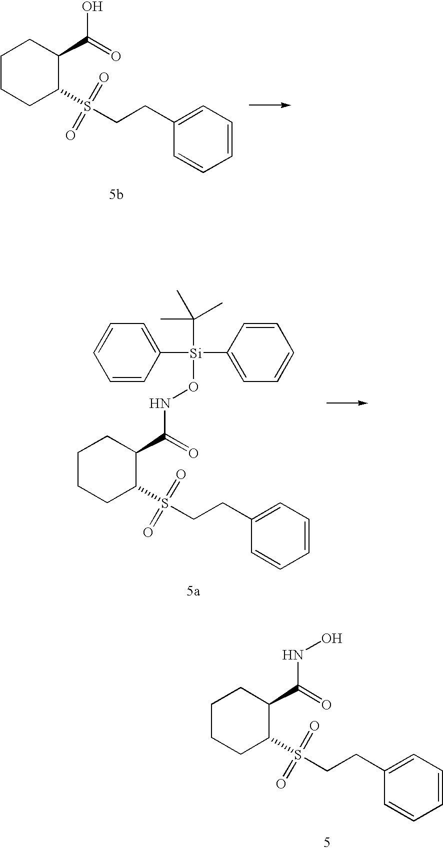

The compound of Example 5 was prepared in the same manner as Example 1, from compound 5a. Purification was performed by column chromatography (ethyl acetate/trace Acetic acid), which afforded a white solid (42%). ¹H NMR (DMSO-d₆): δ 8.93 (1H, s), 7.36-7.35 (5H, m), 3.55-3.50(1H, m), 3.40-3.25 (2H, m), 3.10-2.95 (3H, m), 2.50-2.35 (1H, m), 2.20-2.10 (1H, m), 1.80(1H,bs), 1.75-1.70 (1H, m),1.55-1.05 (4H, m). HRFABMS Calcd for C₁₆H₂₁NO₄S: 312.1269, found 312.1280. Anal. Calcd for C₁₆H₂₁NO₄S: C,57.86; H,6.80; N,4.SO; S,10.30. Found, C, 57.77, H, 6.84; N, 4.51; S,10.20.

### Preparation of Compound 5a: trans-2(2-Phenylethanesulfonyl-cyclohexane carboxylic Acid (O-Dimethyl-tert-butylsilyl)-hydroxyamide

This compound was prepared in the same manner as compound 1a in Example 1, providing a colorless oil (79%), which was used without further purification.

### Preparation of Compound 5b: trans-2-(2-Phenylethanesulfonyl)-cyclohexane carboxylic Acid

This compound was prepared in the same manner as compound 3b in Example 3, from trans-2-(2-phenylethanesulfanyl)-cyclohexanecarboxylic acid in 18 hours to give a solid (70%) that was used without further purification. ¹H NMR (CDCl₃): δ 7.35-7.15 (5H, m), 3.30-3.10 (5H, m),2.90-2.75 (IH, m), 2.30-2.10 (2H, m), 1.95(1H, m),1.80-1.50(4H, m), 1.30(2H, m).

### Example 6

### trans-2-(Biphenyl-4-yl-ethylsulfanyl)cyclohexanecarboxylic Acid Hydroxyamide or (1S,2R)-2-[(2-biphenyl-4-ylethyl)sulfonyl]-N-hydroxycyclohexanecarboxamide

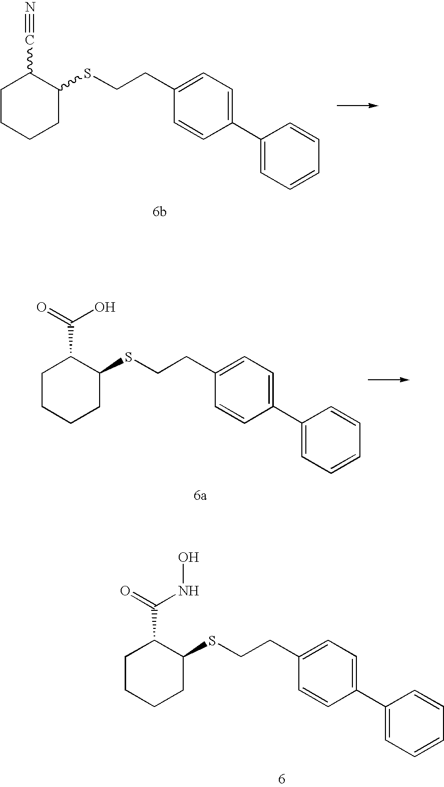

The compound was prepared from compound 6a in the same fashion as Example 1. Upon attempted purification of the silylated hydroxamate by column chromatography (15-30% ethyl acetate/hexane), the deprotected title product had eluted instead as a white solid (49%). ¹H NMR (CDCl₃): δ 8.25 (1H, bs), 7.59-7.25 (9H, m), 2.90-2.79 (4H, m), 2.15 (1H, bs), 1.95-1.60 (7H, m), 1.35-1.15 (2H, m). HRFABMS. Calcd for C₂₁H₂₅O₂SNNa: 378.1504. Found 378.1512. Anal. Calcd for C₂₁H₂₅O₂SN: C, 70.95; H, 7.09; N, 3,94; S, 9.02. Found C, 70.68; H, 7.06; N, 3.90; S. 9.21.

### Preparation of Compound 6a: trans-2-(Biphenyl-4-yl-ethanesulfanyl)-cyclohexane carboxylic Acid

The present compound was prepared in the same manner as compound 3b in Example 3, from compound 6b (from Example 6; 0.42 mmol) with 85% H₃PO₄ (6 mL) and dioxane (4 mL) in place of H₂SO₄, a temperature of 135° C., and time of 5 days. The compound was purified by column chromatography (30-50% ethyl acetate/hexanes) to give a solid (26%), which was used without further purification. HRFABMS. Calcd for C₂₁H₂₅O₂SNNa: 341.1575. Found 341.1568.

### Preparation of Compound 6b cis/trans-2-(Biphenyl-4-yl-ethanesulfanyl)-cyclohexanecarbonitrile

The present compound was prepared in the same manner as compound 3c in Example 3, from compound 1e (from Example 1) after 21 hours stirring to give a 1:1 isomeric mixture (total yield 53%). The isomers were separated by column chromatography (10-20% ethyl acetate/hexane). Cis-isomer: 1H NMR (CDCl₃): δ 59-7.24 (9H, m), 3.09 (1H, m), 2.93-2.90 (4H, m), 2.72 (1H, m), 2.05(1H, m), 1.95-1.56 (6H, m), 1.35 (1H, m). Trans-isomer: ¹H NMR (CDCl₃): δ 7.60-7.29 (9H, m), 2.97 (4H, m), 2.73 (1H, m), 2.56 (1H, m), 2.10 (2H, m), 1.70-1.56 (4H, m), 1.50-1.30(2H, m). Anal for mixture C₂₁H₂₃NS: C, 78.46; H, 7.21; N, 4.36; S, 9.97. Found C, 78.36, H, 7.21; N, 4.40; S, 9.88.

### Example 7

### cis-2-(Biphenyl-4-yl-ethanesulfonyl)cyclohexanecarboxylic Acid Hydroxamate

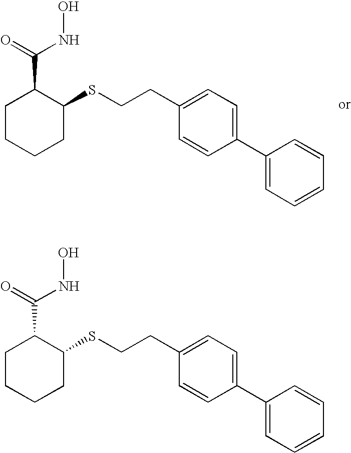

The present compound was prepared in the same fashion as in Example 1, from compound 7a. The compound was purified by dissolution in methanol, evaporation to near dryness, followed by trituration with minimal ethyl acetate, and washing with diethyl ether to give a cream-colored solid (81%). ¹H NMR (CDC1₃): δ 10.60 (1H, s), 8.82 (1H, s), 7.71-7.40 (9H, m), 3.40-3.25(8H, m), 3.00 (1H, m), 2.75(1H, m), 2.50(1H, m),1.90(1H, m), 1.60(1H, m), 1.35(1H, m). Anal. Calcd for C₂₁H₂₅O₄S. 0.25 H₂O: C, 64.34; H, 6.56; N, 3.57, S, 8.18. Found: C, 64.38; H, 6.49, N, 3.47; S,7.91. HRFABMS. Calcd for C₂₁H₂₅O₄S Na 410.1402. Found: 410.1410.

### Preparation of Compound 7a: cis-2-(Biphenyl-4-yl-ethanesulfonyl)-cyclohexane carboxylic Acid (O-Dimethyl-tert-butylsilyl)-hydroxamate

The compound was prepared in the same fashion as in Example 1, from compound 7b. Purification was performed by column chromatography (ethyl acetate), and afforded a white foamy solid (64%), which was used without further purification.

### Preparation of Compound 7b: cis-2-(biphenyl-4-yl-ethanesulfonyl)-cyclohexane carboxylic acid

Prepared in the same fashion as compound 3b in Example 3, from the cis isomer of compound 7c after 4 days stirring. Purification was performed by column chromatography (30-50% ethyl acetate/hexanes), which gave viscous oil (68%). 1H NMR (CDCl₃): δ 7.59-7.25 (9H, m), 3.35 (1H, bs), 2.92-2.74 (4H, m), 2.00-1.90 (1H, m), 1.78 (6H, m), 1.45(1H, m), 1.25 (1H, m). Anal. Calcd for C₂₁H₂₄O₂S: C, 74.08; H, 7.10; S. 9.42. Found: C, 73.85; H, 7.12; S, 9.54.

### Preparation of Compound 7c: cis-2-(biphenyl-4-yl-ethanesulfanyl)-cyclohexane carboxylic acid

The compound was prepared in the same fashion as compound 4b in Example 4, from compound 6b (from Example 6), after 18 hour stirring. Purification was performed by column chromatography (ethyl acetate/trace Acetic acid) gave a solid (62%), which was used without further purification.

Other representative examples of hydroxamate MMP inhibitors useful in the methods of the present invention for decreasing or preventing HCV viral replication activity include the following compounds:

### Example 8

### methyl 4-({N-[(2S)-4-(hydroxyamino)-4-oxo-2-phenylbutanoyl]-L-leucyl}amino)benzoate

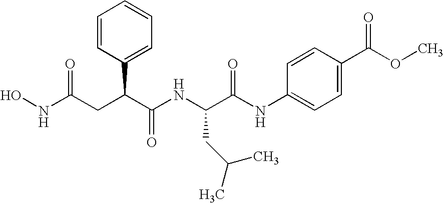

The compound was prepared in similar fashion as described in WO 00/04892.

### Example 9

### 4-acetyl-N-hydroxy-1-[(4-phenoxyphenyl)sulfonyl]piperazine-2-carboxamide

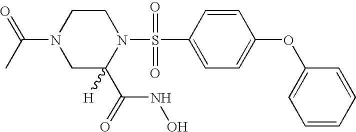

The compound was prepared in the same fashion as the compound of Example 4 of U.S. Pat. No. 5,753,653.

### Example 10

### tert-butyl (3R)-3-[(hydroxyamino)carbonyl]-4-{[4-(pyridin-2-yloxy)phenyl]sulfonyl}piperazine-1-carboxylate

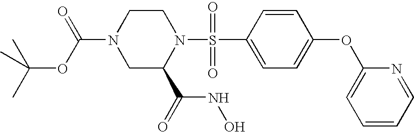

The compound was prepared in the similar fashion as the compound of Example 1 of U.S. Pat. No. 5,753,653.

### Example 11

### (2R)-1-{[4-(4-fluorophenoxy)phenyl]sulfonyl}-N-hydroxy-4-(methylsulfonyl)piperazine-2-carboxamide

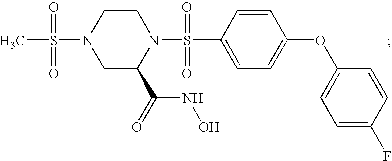

The compound was prepared in the same fashion as the compound of Example 11 of U.S. Pat. No. 5,753,653.

### Example 12

### (3S)—N-hydroxy-2,2-dimethyl-4-{[4-(pyridin-4-yloxy)phenyl]sulfonyl}thiomorpholine-3-carboxamide

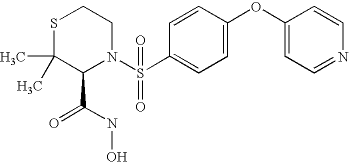

The compound was prepared in the same fashion as the compound of Example 15 of U.S. Pat. No. 5,753,653.

### Example 13

### N-hydroxy-1{[4-(pyridin-4-ylthio)phenyl]sulfonyl}piperidine-2-carboxamide

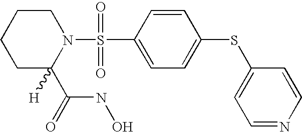

The compound was prepared in similar fashion as described in U.S. Pat. No. 6,153,757.

### Example 14

### N²-{[4-(4-bromophenoxy)phenyl]sulfonyl}-N¹-hydroxy-3{[(5-methylisoxazol-3-yl)methyl]thio}-D-valinamide

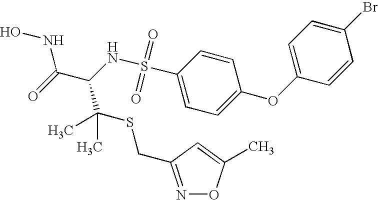

The compound was prepared in the same fashion as the compound of Example 1(b) of U.S. Pat. No. 5,985,653.

The above examples include optically pure compound and enantiomeric mixtures thereof.

### HCV Replicon Assay

All compounds were tested in an HCV reporter replicon assay. Briefly, a reporter replicon containing Huh-7 hepatoma cells was grown in DMEM (Invitrogen, Carlsbad, Calif.) and seeded in 96-well black wall, clear-bottom plates (Costar®; Coming Incorporated). Cells were allowed to settle at 37° C., 5% CO₂ for 30 minutes. The compounds were serially diluted in separate 96 well plates and 100 ml of each concentration was added to the appropriate well in triplicate. The plates are incubated at 37° C., 5% CO₂ for three days.

Following three days of incubation, the media was aspirated from the wells and cells are washed with 100 μl PBS. After removing the PBS, 20 μl of 1× Passive Lysis Buffer (Promega Corp., Madison, Wis.) is added to each well, and the cells are allowed to lyse at room temperature for 15 minutes. Antiviral activity and cytotoxicity is measured following lysis using the dual luciferase kit (Promega Corp., Madison, Wis.). The percent antiviral inhibition and percent cytotoxicity for each concentration is calculated after subtracting the background values of media only wells from wells containing cells, and subtratcting 100 from the percent ratio of the value in the compound well to the cell only control well. This results in the generation of effective concentrations of compounds where 50% antiviral inhibition is observed (EC₅₀) and 50% cytotoxic concentration (CC₅₀) of compounds.

EC₅₀ data as determined for exemplary compounds of the invention are presented in Table 1 below.

TABLE 1

| | Extended 8-pt Assay | | | | 7-pt Assay | | | | | | |
|---|---|---|---|---|---|---|---|---|---|---|---|
| | EC₅₀ | CC₅₀ | | Sol. | EC₅₀ | CC₅₀ | CC₅₀/ | | | Sol. | |
| Ex. | (μM) | (μM) | Tl | (μM) | (μM) | (μM) | EC₅₀ | Tl | Sol. | (μM) | A |
| 1 | 1.6 | 31 | 19 | >320 | 2.1 | 32 | 15 | 15. | 100 | <320 | + |
| 3 | 1.9 | 211 | 109 | >320 | 2.5 | 320 | 130 | >130 | 320 | >320 | + |
| 5 | 0.31 | >320 | >103 | >320 | 0.24 | >320 | 1333 | >1333 | 320 | >320 | + |
| | | | 2 | | (exp) | | | | | | |
| 6 | 0.05 | 99 | 1980 | >320 | 0.034 | 111 | 3265 | >3265 | 320 | >320 | + |
| | | | | | (exp) | | | | | | |
| 8 | 1.2 | >320 | >263 | <320 | 1.5 | >320 | 218 | >218 | 32 | <100 | + |
| 9 | 0.018 | 79 | 4389 | >320 | 0.097 | 81 | 835 | 835. | 320 | >320 | + |
| | | | | | (exp) | | | | | | |
| 10 | 0.19 | 224 | 1178 | >320 | 0.15 | 294 | 1937 | 1937. | 320 | >320 | + |
| | | | | | (exp) | | | | | | |
| 11 | 0.049 | 15 | 306 | >320 | 0.027 | 12 | 444 | 444. | 320 | >320 | + |
| | | | | | (exp) | | | | | | |
| 12 | 0.26 | >320 | >123 | >320 | 0.36 | 320 | 880 | 888. | 320 | >320 | + |
| | | | 0 | | | | | | | | |
| 13 | 0.35 | 211 | 602 | >320 | 0.44 | 293 | 667 | 666. | 320 | >320 | + |
| 14 | 1.9 | 41 | 21 | <320 | 1.57 | 78 | 49 | 49. | 320 | >320 | |

Referring to Table 1, Ex. is defined as example #. Sol. is defined as solubility. A is defined as activity.

While the invention has been described in terms of various preferred embodiments and specific examples, the invention should be understood as not being limited by the foregoing detailed description, but as being defined by the appended claims and their equivalents.

We claim:

1. A method of decreasing or preventing HCV viral replication activity comprising contacting an HCV polymerase with a therapeutically effective amount of a hydroxamate MMP inhibitor.

2. A method according to claim 1, wherein the hydroxamate MMP inhibitor is administered orally or intraveously.

3. A method of treating a condition that is mediated by HCV polymerase in a patient, comprising administering to said patient a pharmaceutically effective amount of a hydroxamate MMP inhibitor.

4. A method according to claim 1 further comprising the step of targeting MMP inhibition as a means of treating indications caused by HCV infections.

5. A method according to claim 1 further comprising the step of targeting viral or cellular targets identified by using MMP inhibitors for treating indications caused by HCV infections.

6. A method according to claim 1 further comprising the step of identifying cellular or viral pathways interfering with the functioning of HCV polymerase which could be used for treating indications caused by HCV infections by administering an MMP inhibitor.

7. A method according to claim 1 further comprising the step of using MMP inhibitors for carrying out gene profiling experiments for monitoring the up or down regulation of genes for the purpose of identifying inhibitors for treating indications caused by HCV infections.

8. A pharmaceutical composition for the treatment of Hepatitis C virus (HCV) in a mammal comprising an amount of hydroxamate MMP inhibitor that is effective in treating HCV and a pharmaceutically acceptable carrier.

9. A method according to claim 1 utilizing a hydroxamate MMP inhibitor of the formula I: 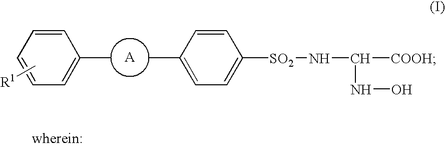
A is a bond, CONH, or 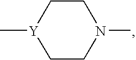
wherein Y is CH or N;
R¹ is alkyl, aryl, halo, amino, substituted or distributed amino, or alkoxy;
and the pharmaceutically acceptable salts thereof.

10. A method according to claim 1 utilizing a hydroxamate MMP inhibitor of the formula (II):

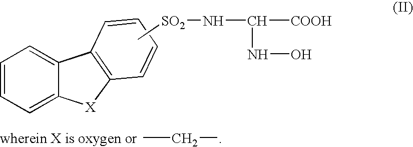

11. A method according to claim 1 wherein the hydroxamate MMP inhibitor is selected from the group consisting of:
2-(2-Phenylethyl)benzoic acid N-hydroxyamide;
2-(Propylthio)-pyridine-3-N-(hydroxy)carboxamide;
[4-(N-Hydroxyamino)-2R-isobutyl-3S-((thien-2-yllthio)methyl)succinyl]-L-phenylalanine-N-methylamide;
N-Hydroxy-5-phenylpentanamide;
2-(Phenyl-2-ethyl)pyridine-3-N-hydroxycarboxamide;
2-(Thiobenzyl)benzoic acid N-hydroxy amide;
6-Biphenyl-4-yl-[2,2-dimethyl-1-(pyridin-4-ylcarbamoyl)-propylcarbamoyl]-hexanoic acid, N-hydroxyamide;
3R(6-(4-Biphenyl)-3-(N-benzylcarbamoyl))-hexanoic acid N-hydroxyamide;
2-Benzylsulfonyl-cyclopent-1-ene-carboxylic acid hydroxamide;
2-Benzylsulfonyl-cyclohex-1-enecarboxylic acid hydroxyamide;
6-Benzylsulfonyl-cyclohex-1-enecarboxylic acid hydroxyamide;
1-(N-Hydroxy)-3-(2-bibenzyl)urea;
3R-(6-(4-Biphenyl)propyl)-N-(3-methylpyridinecarbamoyl)-hexanoic acid N-hydroxy-amide;
4-(2-{[5-Hydroxyamino-3-(3-phenyl-propyl)-3,4-dihydro-2-H-pyrrole-3-carbonyl]-amino}-4-methyl-pentanoylamino)benzoic acid methyl ester;
5-Hydroxyamino-3-(3-phenyl-propyl)-3,4-dihydro-2-H-pyrrole-3-carboxylic acid (2-cyclohexyl-1-methylcarbamoyl-ethyl) amide;
4-(2-{[5-Hydroxyamino-3-(3-pentyl)-3,4-dihydro-2-H-pyrrole-3-carbonyl]-amino}-4methyl-pentanoylamino)benzoic acid methyl ester;
6-Biphenyl-4-yl-3-(R)-(2-hydroxy-1-hydroxymethyl-ethylcarbamoyl)-hexanehydroxamic acid;
6-Biphenyl-4-yl-3(R)-(1(S)-hyroxymethyl-2,2-dimethyl- propylcarbamoyl)-hexanehydroxamicacid;
2-(Biphenyl-4-ylsulfonyl)-cyclohex-1-enecarboxylic acid hydroxyamide;
6-(Biphenyl-4-ylsulfonyl)-cyclohex-1-enecarboxylic acid hydroxyamide;
2-Phenethylsulfanyl-cyclohex-I-enecarboxylic acid hydroxyamide;
2-Benzylsulfanyl-cyclohexancarboxylic acid hydroxamide;
trans-2-Benzylsulfanyl-cyclohexancarboxylic acid hydroxamide;
trans-2-(Biphenyl-4-yl-methylsulfanyl)-cyclohexancarboxylic acid hydroxamide;
6-Biphenyl-4-yl-3-(R)-(1-hydroxymethyl-2-(S)-(1H-imidazol-4-yl)-ethylcarbamoyl)-hexanehydroxamic acid;
N-Hydroxy-2-[2-Oxo-3-(3-phenyl-propyl)-tetrahydro-furan-3-yl]-acetamide;
trans-2-(4-Phenoxy-benzylsulfanyl)-cyclohexancarboxylic acid hydroxamide;
2-(4-Indol-1-yl-benzylsulfanyl)--cyclohexancarboxylic acid hydroxamide;
2-(3-Biphenyl-4-yl-propyl)-N4-hydroxy-N1-(2,4,5-trihydroxy-6-hydroxymethyl-tetrahydro-pyran-3-yl)-succinamide;
2-(2-Biphenyl-4-yl-ethylsulfanyl)-cyclohexane carboxylic acid hydroxyamide;
2-(3-Biphenyl-4-yl-propyl)-N4-hydroxy-N1-(2-hydroxy-cyclohexyl)-succinamide;
6-Biphenyl-4-yl-3-(1-hydroxyimino-ethyl)-hexanoic acid hydroxyamide;
3-(R)-(2-Hydroxy-1-(S)-(1H-imidazol-4-yl)-ethylcarbamoyl)-6-(4-(2-methyl-thiazol-4-yl)-phenyl)-hexanehydroxamic acid;
6-Biphenyl-4-yl-3-(3-hydroxy-piperidine-1-carbonyl)-hexanoic acid-hydroxyamide;
1-(4-Methoxy-benzenesulfonyl)-piperidine-2-carboxylic acid hydroxamide;
1-1-[4-Bromo-phenoxy)-benzenesulfonyl)-piperidine-2-carboxylic acid hydroxyamide;
N-(1-benzyl-2-hydroxy-ethyl)-N4-hydroxy-2-isobutyl-succinamide;
6-Biphenyl-4-yl-3(R)-2(S)-hydroxy-(1(S)-hydroxymethyl-2,2-dimethyl-propylcarbamoyl)-hexanoic hydroxamic acid;
6-Biphenyl-4-yl-3-(2-hydroxy-1hydroxmethyl-propylcarbamoyl)-hexanoic hydroxamic acid;
trans-2-(3-Biphenyl-4-yl-propyl)-cyclohexane carboxylic acid hydroxyamide;
1-[4-Biphenyl-4-yloxy)-benzenesulfonyl)-piperidine-2-carboxylic acid hydroxamide;
1-(4-Phenoxy-benzenesulfonyl)-piperidine-2-carboxylic acid hydroxamide;
6-Biphenyl-4-yl-3-(R)-(1-(S)-hydroxymethyl-2-(3-pyridyl)-ethylcarbamoyl)-hexanehydroxamic acid;
6-Biphenyl-4-yl-2S-hydroxy-3R-(1S-hydroxymethyl-3-methylsulfanyl-propylcarbamoyl)-hexanoic hydroxamic acid;
1-[-[4-(4-Bromo-phenoxy)-benzenesulfonyl]-4-(tertbutoxycarbonyl)-piperazine-2-carboxylic acid hydroxyamide;
1-[4-(4-Bromo-phenoxy)-benzenesulfonyl]-piperazine-2-carboxylic acid hydroxyamide;
4-Acetyl-1-[4-phenoxy-benzenesulfonyl]-piperazine-2-carboxylic acid, N-hydroxyamide;
1-(Diphenylphosphinic)-piperidine-2-carboxylic acid hydroxamide;
6-Biphenyl-4-yl-3-(R)-(2-oxo-I-tetrahydrofuran-3-(S)-ylcarbamoyl)-hexane hydroxamic acid;
1-[-[4-(4-Bromo-phenoxy)-benzenesulfonyl]-4-methyl-piperazine-2-carboxylic acid N-hydroxyamide;
4-(4-Methoxy-benzenesulfonyl)-thiomorpholine-3-carboxylic acid hydroxyamide;
3-(Diphenylphosphinic)-propanoic acid hydroxyamide;
1-[4-(4-Chlorophenoxy)benzenesulfonyl]-thiomorpholine-3-carbamoyl)piperazine-2-carboxamide;
4[4-Phenoxy-benzenesulfonyl]-piperazine-2-carboxylic acid, N-hydroxyamide;
4[4-Phenoxy-benzenesulfonyl]-thiomorpholine-3-carboxylic acid N-hydroxyamide;
3[2-Biphenyl-4-yl-ethylsulfanyl]-tetrahydro-pyran-4-carboxylic acid N-hydroxyamide;
1-[4-Phenoxy-benzenesulfonyl]-4-methyl-piperazine-2-carboxylic acid N-hydroxyamide;
6-Biphenyl-4-yl-3-(R)-(2-oxo-azepan-3-(S)-ylcarbamoyl)-hexane hydroxamic acid;
4-(1H-Indole-2-sulfonyl)-thiomorpholine-3-carboxylic acid hydroxyamide;
1-(Methyl-phenylphosphinic)-piperidine-2-(R)-carboxylic acid hydroxamide;
1-(1,3-Dihydro-isoindole-2-sulfonyl)-piperidine-2-carboxylic acid hydroxamide;
4-Methyl-1-(4-(4-chlorophenyl)benzenesulfonyl)-N-hydroxy-2R-piperazinecarboxamide hydrochloride;
1-[4-Chlorophenoxybenzenesulfonyl]-N-hydroxy-2R-piperazinecarboxamide;
2-(3-Phenyl-propylsulfonyl)-cyclohexane carboxylic acid hydroxamide;
1-(Pyrolidine-1-sulfonyl)-piperidine-2-carboxylic acid hydroxyamide;
1-(Piperidine-1-sulfonyl)-piperidine-2-carboxylic acid hydroxyamide;
4-[-[4-Bromo-phenoxy-benzenesulfonyl]-oxothiomorpholine-3-carboxylic acid-N-hydroxyamide;
1-[4-(4-Methoxy-phenylsulfanyl)-benzenesulfonyl]-piperdine-2-carboxylic acid hydroxyamide;
1-[4-(4-Cyano-phenoxy)-benzenesulfonyl]-4-(tert-butoxycarbonyl)-piperazine-2-carboxylic acid N-hydroxyamide;
6-Oxo-3-(4-phenoxy-benzenesulfonyl)-hexahydro-pyrimidine-4-carboxylic acid hydroxamate;
4-(t-Butoxycabonyl)-1-(4-(pyridin-2-yl)oxybenzensulfonyl)-N-hydroxy-piperazine-2-carboxamide;
4-[(4-Fluorophenoxy)-benzenesulfonyl]-thiomorpholine-3--carboxylic acid N-hydroxyamide;
4-[4-(Fluoro-phenoxy)-benzenesulfonyl]-oxothiomorpholine-3-carboxylic acid N-hydroxyamide;
4-(4-Butoxy-benzenesulfonyl)-thiomorpholine-3-carboxylic acid hydroxyamide;
4-(4-Butoxy-benzenesulfonyl)-1-oxothiomorpholine-3-carboxylic acid hydroxyamide;
1-[4-(4-Fluorophenyl)benzenesulfonyl]-4-(tert-butoxycarboxy)2R-piperazine-2-carboxylic acid hydroxyamide;
1-((4-(4-Chlorophenyl)-piperazine)--I-sulfonyl)-piperidine-2carboxylic acid hydroxamide;
cis-2-Phenethylsulfanyl-cyclohexanecarboxylic acid hydroxyamide;
1-[-[4-(4-Fluorophenyl)benzenesulfonyl)-N-hydroxy-2R-piperazinecarboxamide hydrochoride;
1-(Diphenylphosphinic)-pyrolidine-2(R)-carboxylic acid hydroxyamide;
trans-2-Phenethylsulfonyl-cyclohexanecarboxylic acid hydroxyamide;
1-[4-(4-Flourophenyl)-piperazine-1-sulfonyl]-piperidine-2-carboxylic acid hydroxamide;
1-1-[4-(4-Fluorophenylsulfanyl)-benzenesulfonyl]-piperidine-2-carboxylic acid hydroxyamide;
4-1-[4-(Bromo-phenoxy)-benzenesulfonyl]-2,2-dimethyl-1-oxo-thiomorpholine-3-carboxylic acid hydroxyamide;
1-(Pyrrolidine-1-carbonyl)-pyrrolidine-2(R)-carboxylic acid hydroxyamide;
R-4-[4-(Bromophenoxy)-benzenesulfonyl]-2,2-dimethyl-1-oxo-thiomorpholine-3-carboxylic acid hydroxyamide;
4-(Ethoxycarbonyl)methyl-1-(4-(4-chlorophenyl)benzenesulfonyl)-N-hydroxy-2R-piperazinecarboxamide hydrochloride;
1-Phenethylcarbamoyl-pyrrolidine-2-(R)-carboxylic acid hydroxyamide;
1-(4-Benzyl-piperazine-1-sulfonyl)-piperidine-2-carboxylic acid hydroxyamide;
3(S)—N-Hydroxy-4-(4-(pyridin-4-yl) oxybenzenesulfonyl)-2, 2- dimethyl-tetrahydro-2H-1,4-thiazine-3-carboxamide;
2(R)-4-Methyl-1-(4-(4-fluorophenyl)benzenesulfonyl)-N-hydroxy-piperazine-2-carboxamide;
1-((2-Pyridyl)-4-piperazine-1-sulfonyl)-piperdine-2-carboxylic acid hydroxyamide;
1-1-[4-(Pyridin-4-ylsulfamyl)-benzenesulfonyl]-piperdine-2-carboxylic acid hydroxyamide;
N-(4-Phenoxy-benzenesulfonyl)-D-tert-leucine-N-hydroxyamide;
2,2-Dimethyl-4-[4-(pyridin-2-yloxy)-benzenesulfonyl]-thiomorpholine-3-carboxylic acid hydroxyamide;
N-1-[4-(4-Fluorophenoxyl)benzenesulfonyl)-D-tert-leucine, N-hydroxyamide;
3(R)—N-Hydroxy-4-(4-(pyridin-4-yl) oxybenzenesulfonyl)-2,2-dimethyl-tetrahydro-2H-1,4-thiazine-3-carboxamide hydrochloride;
2-[4-(4-Chloro-phenoxy)-benzenesulfonylamino]-N-hydroxy-3,3-dimethyl-butyramide;
3(R)—N-Hydroxy-4-(4-(fur-3-yl) phenoxybenzenesulfonyl)-2,2-dimethyl-tetrahydro-2H-1,4-thiazine-3-carboxamide;
2-1-[4-(Pyridin-2-yl-oxy)-benzenesulfonylamino]-N-hydroxy-3,3-dimethyl butyramide;
2-(2-Biphenyl-4-yl-ethylsulfonyl)-cyclohex-1-ene-carboxylic acid hydroxyamide;
6-(2-Biphenyl-4-yl-ethyl sulfonyl)-cyclohex-I-ene-carboxylic acid hydroxyamide;
N-(4-Phenoxy-benzenesulfonyl)-3,3-dimethyl-S-(methylthio)-D-cysteine, N-hydroxyamide;
(4-Phenoxy-piperidine-1-sulfonyl)-piperdine-2-carboxylic acid hydroxyamide;
N-(4-[4-Chlorophenoxy]-benzenesulfonyl)-3,3-dimethyl-S-(methylthio)-D-cysteine, N-hydroxyamide;
N-(4-[4-Chlorophenoxy]-benzenesulfonyl)-3,3-dimethyl-S-(methylsulfoxy)-D-cysteine, N-hydroxyamide;
cis-2-(2-Phenyl-ethanesulfonyl)-cyclohexanecarboxylic acid hydroxyamide;
3(R)—N-Hydroxy-4-(4-(imidazol-1-yl) phenoxybenzenesulfonyl)-2,2-dimethyl-tetrahydro-2H-1,4-thiazine-3-carboxamide;
3(R)—N-Hydroxy-4-(4-(pyridin-4-yl) oxybenzenesulfonyl)-2,2-dimethyl-tetrahydro-2H-1,4-thiazine-3-carboxamide;
4-1-[2-(2-Hydroxycarbamylmethyl-5-phenyl-pentanoylamino)-4-methyl-pentanoyl]-benzoic acid methyl ester;
trans-2-(2-Phenyl-ethanesulfonyl)-cyclohexanecarboxylic acid hydroxyamide;
3,3-Dimethyl-2-(4-phenoxy-phenylsulfanylmethyl)-butyric acid, N-hydroxyamide;
2-(2-Biphenyl-4-yl-ethanesulfonyl)-cyclohexanecarboxylic acid hydroxamate;
2-[-[4-(4-Chlorophenyl)-piperazine-1-sulfonylamino]-3-methyl-3-(pyridin-2ylmethylsulfanyl)-butyric acid N-hydroxyamide;
3,3-Dimethyl-2-(4-phenoxy-phenylsulfonylmethyl)-butyric acid, N-hydroxyamide;
2(R)-[4-(4-Fluoro-phenoxy)benzenesulfonylamino]-3-methyl-3-(pyridin-2-yl sulfanyl)-butyric acid, hydroxyamide;
3(R)—N-Hydroxy-4-(4-(-((pyridin-4-yl)methyl) oxybenzenesulfonyl)-2,2-dimethyl-tetrahydro-2H-1,4-thiazine-3-carboxamide;
1-1-[4-(4-Chloro-phenoxy)-benzenesulfonyl]-4-(l-methyl-1H-imidazole-4-sulfonyl)-piperazine-2-carboxylic acid hydroxamide;
1-[4-(Pyridin-2-ylsulfanyl)-piperidine-I-sulfonyl]-piperidine-2-carboxylic acid hydroxyamide;
2R-[4-(4-Furan-3-yl-phenoxy)-benzenesulfonylamino]-N-hydroxy-3-methyl-3-(pyridin-4-ylsulfanyl)-butyramide;
trans-2-(2-Biphenyl-4-yl-ethylsulfanyl)-cyclohexanecarboxylic acid hydroxyamide;
N4-(2,2-Dimethyl-1S-hydroxymethyl-propyl)-N1-hydroxy-3R[3-(4-pyridin-4-yl-phenyl)-pyrrol-1-yl]-succindiamide;
1-[4-(4-Fluoro-phenoxy)-benzenesulfonyl)]-3,3-dimethyl-5-oxo-piperazine-2-carboxylic acid hydroxyamide;
2(R)-[4-(4-Iodo-phenoxy)benzenesulfonylamino]-3-methyl-(pyrdin-3-yl-sulfonyl) butyric acid hydroxyamide;
1-[-[2-(Benzothiazol-2-ylsulfanyl)-piperidine-1-sulfonyl]piperidine-2-carboxylic acid hydroxyamide;
5-[4-(4-Fluoro-phenoxy)-benzenesulfonyl]-4,5,6,7-tetrahydro-3H-imidazolo[4,5,-c]pyridine-6-carboxylic acid hydroxyamide;
1-[4-(Pyridin-4-ylsulfanyl)-piperidine-1-sulfanyl]-piperidine-2carboxylic acid hydroxyamide;
1-[4-(4-Methoxy-phenylsulfamyl)-piperidine-1-sulfonyl]piperidine-2-carboxylic acid hydroxyamide;
2(R)-[4-(4-Methylphenoxy)benzenesulfonylamino]-3-methyl-3-(pyridin-3-yl-sulfonyl) butyric acid hydroxyamide;
1-[4-(4-Methyl-phenylsulfamyl)-piperidine-1-sulfonyl]-piperidine-2-carboxylic acid hydroxamide;
4-Methoxy-benzenesulfonyl)-2,2-dimethyl-thiomorpholine-3-carboxylic acid hydroxyamide;
4-1-[4-(4-Chloro-phenoxy)-benzenesulfonyl]-2,2-dimethyl-thiomorpholine-3-carboxylic acid hydroxyamide;
2(R)-[4-(4-bromo-phenoxy)benzenesulfoxylamino]-3-methyl-3-(pyridin-4-yl-sulfoxide) butyric acid hydroxyamide;
4-(4-Methoxy-benzensulfonyl)-2,2-dimethyl-1-oxo-thiomorpholine-3-carboxylic acid hydroxyamide;
4-4-(4-Chloro-phenoxy)-benzenesulfonyl]-2,2-dimethoxy-1-oxo-thiomorpholine-3-carboxylic acid hydroxyamide;
3(S)-2,2-Dimethyl-4-[4-(pyrdin-4-ylsulfanyl)-benzenesulfonyl]-thiomorpholine-3-carboxylic acid hydroxyamide;
3,3-Dimethyl-N-hydroxy-2R-[-[4(-(pyridin-4-ylsulfanyl)-piperidine-1-sulfonylamino]-butyramide;
N-Hydroxy-2-[-[(4-methylbenzenesulfonyl) amino]acetamide;
[4(-(4-Imidazol-1-yl-phenoxy)-piperidine-I-sulfonyl]-piperidine-2-carboxylic acid hydroxyamide;
1-[4-(4-Imidazol-1-yl-phenylsulfanyl)-piperidine-1-sulfonyl]-piperidine-2-carboxylic acid hydroxyamide;
2(R)-[4-(4-Chloro-benzoyl)-cyclohexanesulfonyl]-piperidine-1-carboxylic acid hydroxyamide;
1(R)-[4-(4-Chloro-benzoyl)-piperidine-1-sulfonyl]-piperidine-2-carboxylic acid hydroxyamide;
1(R)-(4-Pyridin-2-yl-piperazine-1-sulfonyl)-piperidine-2-carboxylic acid hydroxyamide;
1(R)-[4-(4-Imidazol-1-yl-phenoxy)-piperidine-1-sulfonyl]-piperidine-2-carboxylic acid hydroxyamide;
N-Hydroxy-3,3-dimethyl-2R-[4(-(morpholine-4-carbonyl)-piperidine-1-sulfonylamino]-butyramide;
N-Hydroxy-3-methyl-3-(5-methyl-isoxazol-3-yl-methylsulfanyl)-2R-[4-(pyridin-4-ylsulfanyl)-piperidine-sulfonylamino]-butyramide;
N-Hydroxy-2R-[4-(4-imidazol-1-yl-phenoxy)-piperidine-1-sulfonylamino]-3,3-dimethyl-butyramide;
2R-[4-(4-Chloro-benzoyl)-piperazine-1-sulfonylamino]-Nhydroxy-3-methyl-3-methylsulfanyl-butyramide;
N-Hydroxy-3-methyl-3-methylsulfanyl-2R-[4-(pyrdin-4-ylsulfanyl)-piperidine-1-sulfonylamino]-butyramide;
1R,3S,2,2-Dimethyl-1-oxo-4-[-[4(-(pyridin-4-yloxy)-benzenesulfonyl]-thiomorpholine-3-carboxylicacid amide;
and the pharmaceutically acceptable salts thereof.

12. A method according to claim 1 wherein the hydroxamate MMP inhibitor is selected from the group consisting of: 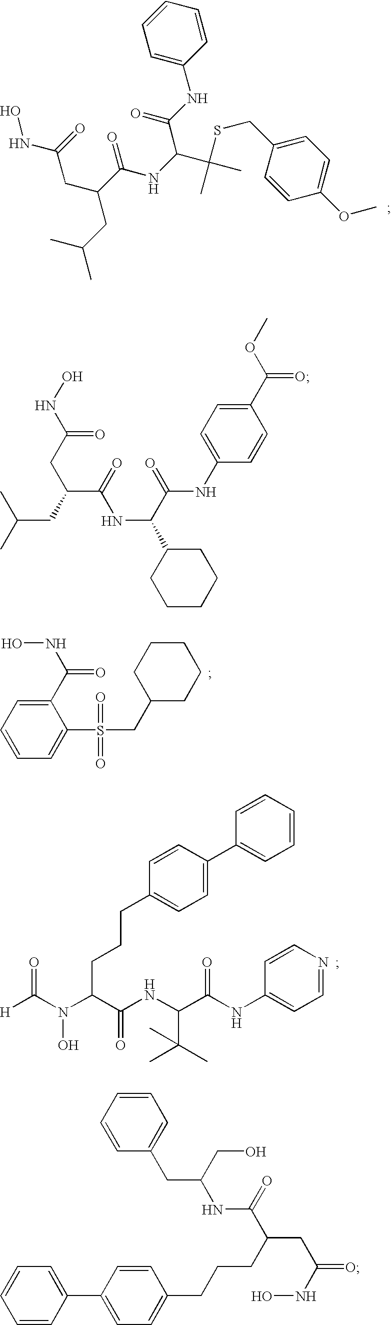 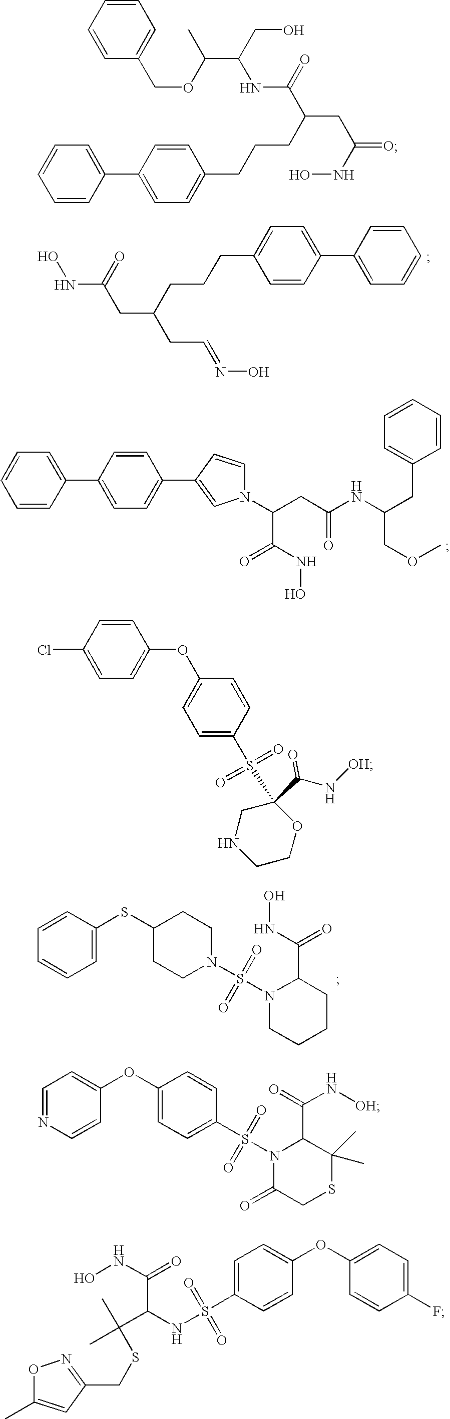
and the pharmaceutically acceptable salts thereof.

13. A method according to claim 1 wherein said hydroxamate MMP inhibitor is of the formula (III): 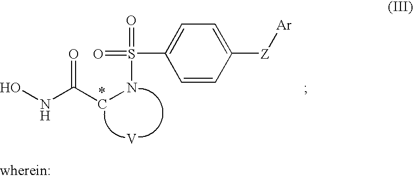
Z is O or S; V is a divalent radical which together with C* and N forms a ring having six ring atoms, where each of said ring atoms other than C* and N independently is unsubstituted or substituted by a suitable substituent, and at least one of said other ring atoms is a heteroatom selected from O, N and S, and the remainder are carbon atoms; and Ar is an aryl or heteroaryl group; or a pharmaceutically acceptable prodrug, salt or solvate thereof.

14. A method according to claim 13 wherein said hydroxamate inhibitor is selected from the group consisting of:
2(R)—N-hydroxy-1-(4-(4-chlorophenoxy)benzenesulfonyl)-4-(methanesulfonyl)-piperazine-2-carboxamide;
2(R)—N-hydroxy-1-(4-(4-fluorophenoxy)benzenesulfonyl)-4-(methanesulfonyl)-piperazine-2-carboxamide;
3(S)—N-hydroxy-4-(4-((pyrid-4-yl) oxy)benzenesulfonyl)-2,2-dimethyl-tetrahydro-2H-1,4-thiazine-3-carboxamide;
and the pharmaceutically acceptable salts thereof.

15. A method according to claim 1 wherein said hydroxamate inhibitor is of the formula (IV): 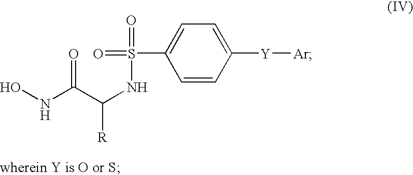
Ar is an aryl group or a heteroaryl group;
R is H, an alkyl group, a cycloalkyl group, a heterocycloalkyl group, an aryl group, a heteroaryl group, or —C(O)R¹, wherein R¹ is hydrogen, an alkyl group, a cycloalkyl group, a heterocycloalkyl group, an aryl group, a heteroaryl group, or —NR² R³, wherein R² and R³ independently are hydrogen, an alkyl group, a cycloalkyl group, a heterocycloalkyl group, an aryl group, or a heteroaryl group;
or the pharmaceutically acceptable salts thereof.

16. A method according to claim 15, wherein said hydroxamate inhibitor is selected from the group consisting of:
2(S)—N-hydroxy-3,3-dimethyl-2-[(4-(4-fluorophenoxy)benzenesulfonyl)-amino]butanamide;
2(S)—N-hydroxy-3,3-dimethyl-2-[(4-(4-chlorophenoxy)benzenesulfonyl)-amino]butanamide;
2(S)—N-hydroxy-3-methyl-3-(pyrid-2-yl)methylsulfanyl-2-[(4-(4-fluorophenoxy)benzenesulfonyl)-amino]butanamide;
2(S)—N-hydroxy-3-methyl-3-(pyrid-2-yl)methylsulfanyl-2-[(4-(4-bromophenoxy)-benzenesulfonyl)-amino]butanamide;
2(S)—N-hydroxy-3-methyl-3-(pyrid-2-yl)methylsulfanyl-2-[(4-(4-iodophenoxy)benzenesulfonyl)-amino]butanamide;
2(S)—N-hydroxy-3-methyl-3-(5-methylisoxazol-3-yl)methylsulfanyl-2-[(4-(4-fluorophenoxy)-benzenesulfonyl)amino]butanamide;
2(S)—N-hydroxy-3-methyl-3-(5-methylisoxazol-3-yl)methylsulfanyl-2-[(4-(4-bromophenoxy)-benzenesulfonyl)amino]butanamide;
2(S)—N-hydroxy-3-methyl-3-(pyrid-2-yl)methylsulfanyl-2-[(4-(4-methylphenoxy)-benzenesulfonyl)amino]butanamide;
2(S)—N-hydroxy-3-methyl-3-(5-methylisoxazol-3-yl)methylsulfanyl-2-[(4-(pyrid-4-yloxy)benzenesulfonyl)-amino]butanamide;
2(S)—N-hydroxy-3-methyl-3-(5-methylisoxazol-3-yl)methylsulfanyl-2-[(4-{(pyrid-4-yl)sulfanyl)benzenesulfonyl)amino]butanamide;
2(S)—N-hydroxy-3-methyl-3-(1H-imidazol-4-yl)methylsulfanyl-2-[(4-(4-bromophenoxy)benzenesulfonyl)-amino]butanamide;
2(S)—N-hydroxy-3-methyl-3-(1-methyl-1H-imidazol-2-yl)methylsulfanyl-2-[(4-(4-bromophenoxy)-benzenesulfonyl)amino]butanamide;
2(S)—N-hydroxy-3-methyl-3-(1-methyl-1H-imidazol-4-yl)methylsulfanyl-2-[(4-(4-bromophenoxy)-benzenesulfonyl)amino]butanamide;
2(S)—N-hydroxy-3-methyl-3-(4-methyl-4H-[1,2,4]-triazol-3-yl)methylsulfanyl-2-[(4-(4-bromophenoxy)-benzenesulfonyl)amino]butanamide;
2(S)—N-hydroxy-3-methyl-3-(1-methyl-4H-[1,2,4]-triazol-3-yl)methylsulfanyl-2-[(4-(4-bromophenoxy)-benzenesulfonyl)amino]butanamide;
2(S)—N-hydroxy-3-methyl-3-methylsulfanyl-2-[(4-(4-chlorophenoxy)benzenesulfonyl)amino]butananamide; and the pharmaceutically acceptable salts thereof.

17. A method according to claim 1 wherein said hydroxamate MMP inhibitor is selected from the group consisting of: 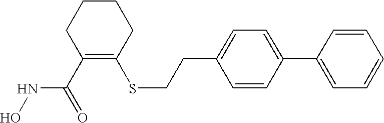
(2-[(2-biphenyl-4-ylethyl)thio]-N-hydroxycyclohex-1-ene-1-carboxamide); 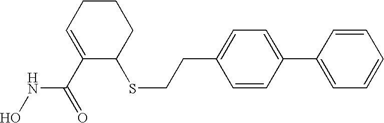
(6-(2-Biphenyl-4-yl-ethylsulfanyl)-cyclohex-1-ene-carboxylic Acid Hydroxyamide); 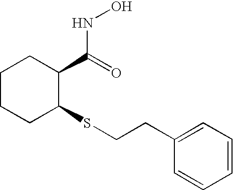
((1R,2R)—N-hydroxy-2-[(2-phenylethyl)thio]cyclohexanecarboxamide); 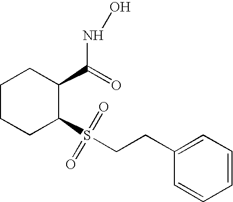
(cis-Phenyl-ethanesulfonyl-cyclohexanecarboxylic Acid Hydroxyamide); 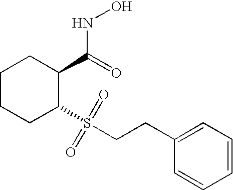
((1S,2R)—N-hydroxy-2-[(2-phenylethyl)sulfonyl]cyclohexanecarboxamide); 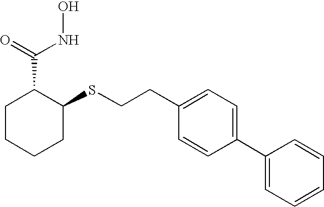
((1S,2R)-2-[(2-biphenyl-4-ylethyl)sulfonyl]-N-hydroxycyclohexanecarboxamide); 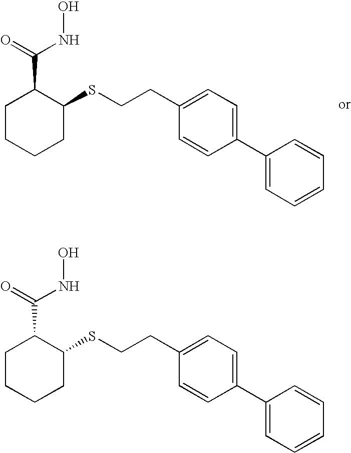
(cis-2-(Biphenyl-4-yl-ethanesulfonyl)-cyclohexanecarboxylic Acid Hydroxamate); 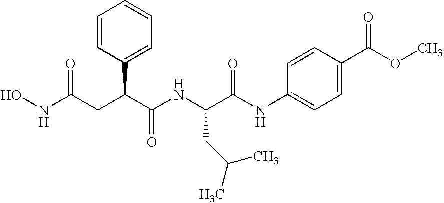
(methyl 4-({N-[(2S)-4-(hydroxyamino)-4-oxo-2-phenylbutanoyl]-L-leucyl}amino)benzoate); 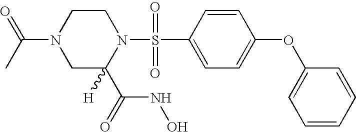
(4-acetyl-N-hydroxy-1-[(4-phenoxyphenyl)sulfonyl]piperazine-2-carboxamide); 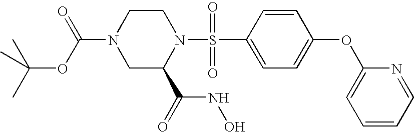
(tert-butyl (3R)-3-[(hydroxyamino)carbonyl]-4-{[4-(pyridin-2-yloxy)phenyl]sulfonyl}piperazine-1-carboxylate); 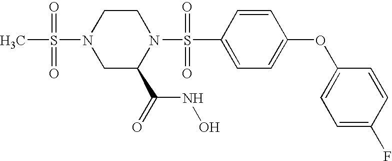
(2R)-1-{[4-(4-fluorophenoxy)phenyl]sulfonyl}-N-hydroxy-4-(methylsulfonyl)piperazine-2-carboxamide); 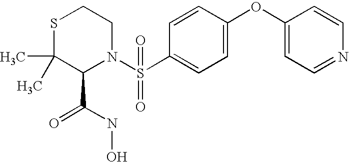
((3S)—N-hydroxy-2,2-dimethyl-4-{[4-(pyridin-4-yloxy)phenyl]sulfonyl}thiomorpholine-3-carboxamide); 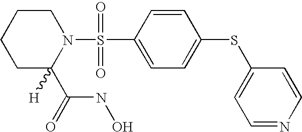
(N-hydroxy-1-{[4-(pyridin-4-ylthio)phenyl]sulfonyl}piperidine-2-carboxamide); 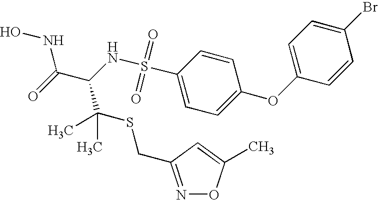
(N²-{[4-(4-bromophenoxy)phenyl]sulfonyl}-N¹-hydroxy-3-{[(5-methylisoxazol-3-yl)methyl]thio}-D-valinamide); the optically pure compound and enantiomeric mixtures thereof; and the pharmaceutically acceptable salts thereof.
